(19) United States
(12) Patent Application Publication
(10) Pub. No.: US 2023/0149553 A1
(43) Pub. Date: May 18, 2023

(54) LIQUID PHARMACEUTICAL FORMULATIONS POLYETHYLENE GLYCOL-BASED PRODRUGS OF ADRENOMEDULLIN AND USE

(71) Applicant: Bayer Aktiengesellschaft, Leverkusen (DE)

(72) Inventors: Florian UNGER, Haan (DE); Stefan Christian SCHNEID, Düsseldorf (DE); Hans-Walter MOTZKUS, Schildow (DE); Carina HAASBACH, Wuppertal (DE)

(73) Assignee: Bayer Aktiengesellschaft, Leverkusen (DE)

(21) Appl. No.: 17/916,778

(22) PCT Filed: Mar. 31, 2021
(86) PCT No.: PCT/EP2021/058427

§ 371 Date: Oct. 3, 2022

(30) Foreign Application Priority Data
Apr. 3, 2020 (WO) 20168068.3

(51) Int. Cl. A61K 47/60; A61K 9/08; A61K 47/54; A61K 9/00

(52) U.S. Cl. CPC A61K 47/60; A61K 9/08; A61K 47/542; A61K 9/0073

(57) ABSTRACT

The present invention relates to novel liquid pharmaceutical formulations, preferably for inhalation, comprising polyethylene glycol (PEG)-based prodrugs of Adrenomedullin (PEG-ADM) and the use thereof for the treatment and/or prevention of acute lung injury/acute respiratory distress syndrome (ALI/ARDS).

The present invention relates to novel pharmaceutical formulations for inhalation comprising polyethylene glycol (PEG)-based prodrugs of Adrenomedullin (ADM) and the use thereof for the treatment and/or prevention of acute lung injury/acute respiratory distress syndrome (ALI/ARDS).

The 52 amino acid peptide hormone ADM is produced in adrenal gland, lung, kidney, heart muscle and other organs. The plasma levels of ADM are in the lower picomolar range. ADM is a member of the calcitonin gene-related peptide (CGRP) family of peptides and as such binds to a heterodimeric G-protein coupled receptor that consists of CRLR and RAMP 2 or 3 (Calcitonin-receptor-like receptor and receptor activity modifying protein 2 or 3). Activation of the ADM receptor leads to intracellular elevation of adenosine 3′, 5′-cyclic monophosphate (cAMP) in the receptor-bearing cells. ADM receptors are present on different cell types in almost all organs including endothelial cells. ADM is thought to be metabolized by neutral endopeptidase and is predominantly cleared in the lung where ADM-receptors are highly expressed [Gibbons C., et al., Mol Endocrinol 21(4), 783-796 (2007)].

Experimental data from the literature suggest that ADM is involved in a variety of functional roles that include, among others, blood pressure regulation, bronchodilatation, renal function, hormone secretion, cell growth, differentiation, neurotransmission, and modulation of the immune response. Moreover, ADM plays a crucial role as autocrine factor during proliferation and regeneration of endothelial cells [Garcia M. A., et al., Expert Opin Ther Targets, 10(2), 303-317 (2006)].

There is an extensive body of evidence from the literature which shows that ADM is indispensable for an intact endothelial barrier function and that administration of ADM to supra-physiological levels exerts strong anti-edematous and anti-inflammatory functions in a variety of inflammatory conditions in animal experiments including sepsis, acute lung injury and inflammation of the intestine [Temmesfeld-Wollbrück B., et al., Thromb Haemost; 98, 944-951 (2007)].

Clinical testing of ADM was so far conducted in cardiovascular indications with a measurable hemodynamic end point such as pulmonary hypertension, hypertension, heart failure and acute myocardial infarction. ADM showed hemodynamic effects in several studies in patients suffering from the aforementioned conditions. However, effects were only short lasting and immediately ceasing after the end of administration. These findings correlated well with the known pharmacokinetic profile of ADM. Pharmacodynamic effects comprised among others lowering of systemic and pulmonary arterial blood pressure and increase of cardiac output [Troughton R. W., et al., Hypertension, 36(4), 588-93 (2000); Nagaya N. and Kangawa K., Peptides, 25(11), 2013-8 (2004); Kataoka Y., et al., J Cardiovasc Pharmacol, 56(4), 413-9 (2010)].

In this respect, compounds described in WO 2013/064508 A1 (“PEG-ADM”) act as slow release prodrugs of ADM with extended duration of pharmacological action as compared to “free” ADM and on the basis of this specific action mechanism exert in vivo sustained anti-inflammatory and hemodynamic effects such as stabilization of endothelial barrier function, and reduction of blood pressure, respectively.

The compounds according to WO 2013/064508 A1 can act systemically and/or locally. For this purpose, they can be administered in a suitable way, for example as a pharmaceutical aerosol intended for inhalation by means of a suitable inhaler device.

The respiratory tract is directly accessible from the outside and thus, an attractive avenue for a targeted administration of therapeutic agents. The basic concept of inhalation is utilized for the treatment of numerous respiratory diseases, owing to the advantages of this approach such as a rapid onset of drug action, high local drug concentration, superior therapeutic selectivity and reduction of side effects [Rau J. L., Respir Care, 50(3), 367-82 (2005)]. The lungs can be accessed by inhalation deposition of different types of pharmaceutical aerosols. Typically, these formulations are composed of particles or droplets (together referred to as “particles” throughout this specification) of a few microns in diameter containing the active ingredient (Hofmann W., J Aerosol Sci, 42(10), 693-724 (2011)]. Having the significant impact of aerosols' physicochemical properties for lung deposition and hence, therapeutic efficacy of the delivered medication in mind, formulation and device design development are currently aiming for a production of optimized aerosols [Dolovich M. and Dhand R., Lancet 377(9770), 1032-45 (2011)].

PEG-ADM is under development as a therapeutic agent for inhalation. The stability of PEG-ADM in solution in liquid state is insufficient for long-term storage as the molecule can be degraded through different pathways such as aggregation, linker separation or disulphide oxidation. In addition, even if a reasonably stable formulation is found, it should be noticed that it must also be suitable for nebulization. Furthermore, it is also of importance that a therapeutically effective concentration is finally delivered to the patient.

A certain concentration of chloride ions (30 mmol) was described as required for inhalation delivery by healthy volunteers to avoid coughing (Eschenbacher W L, Boushey H A, Sheppard D. Alteration in osmolarity of inhaled aerosols cause bronchoconstriction and cough, but absence of a permeant anion causes cough alone. Am Rev Respir Dis 1984; 129:211-215). In addition, severe divergence from isosmotic conditions is not desirable for inhaled solutions. Therefore, a formulation with a relatively high content of sodium chloride was developed, which negatively influenced the thermal characteristics of the product, and would suggest a requirement for very cold storage temperatures.

Kohle and Goswami (Kohle P, Goswami S. Bulk Protein Solution: Freeze-Thaw Process, Storage and Shipping Considerations. Challenges in Protein Product Development, Ed. N. Warne, H C Mahler, AAPS/Springer 2018) studied freezing of solutions containing sodium chloride (NaCl), and reported that complexity of the phase behavior due to inclusion of excipients is apparent from the phase diagram of NaCl. Freezing of a normal saline system (0.9 wt % NaCl in water) causes water to come out of the bulk solution phase as ice crystal, and as a result, the concentration of NaCl increases considerably in the bulk phase. As the temperature of the system is brought down to −21.2° C., the bulk phase experiences a 26-fold increases in salt concentration (23.3 wt %). At this temperature, referred as eutectic point the system is a complex equilibrium mixture of ice, hydrohalite (NaClx2H2O), and saturated solution of saline. Eutectic point also refers to the depressed freezing point of this water/NaCl system. This can be clearly understood from the phase behavior of water/NaCl binary system that storing at −20° C. may not be enough to achieve a truly frozen state.”

Izutsu and Aoyagu (Izutsu K, Aoyagi N. Effect of inorganic salts on crystallization of poly(ethylene glycol) in frozen solutions. International Journal of Pharmaceutics 288 (2005) 101-108) investigated the impact of NaCl on the freezing behavior of solutions containing PEG of a lower chain length (PEG 3000). They reported that NaCl addition significantly reduced the melting endotherm of the solution to lower temperatures. The soluted remained amorphous in the freeze concentrate, except for high NaCl concentrations at which a fraction of NaCl crystallized and the remaining fraction remained in the freeze concentrate. The results suggest that the crystallinity of a solute is determined by complex interplay among the co-solutes in the frozen solution.

Izutsu and Kojima (Izutsu K, Kojima S. Freeze-Concentration Separates Proteins and Polymer Excipients into Different Amorphous Phases. Pharmaceutical Research, Vol. 17, No. 10, 2000) investigated freeze concentration effects on protein and polymer systems and found separation into different amorphous phases. The addition of NaCl resulted in a significant reduction of the glass transition temperature of the maximally freeze-concentrated solute for solutions containing 40 kDa PVP and ovalbumin. Their results strongly suggest that NaCl separates the amorphous ovalbumin and PVP 40k combination into ovalbumin-rich (without apparent Tg′) and PVP-rich (Tg′ at −22° C.) phases in a frozen solution. The salt-induced sudden Tg′ change resembles that of the Tg′ splitting previously observed in DEAE—dextran and dextran combinations. The single Tg′ of the polyelectrolyte and nonionic polymer combination was split into two transitions at certain salt (e.g., NaCl) concentrations due to the freeze-induced phase separation. Some polymer combinations can separate in the cooling process since the polymer interactions depend on temperature. Freezing significantly concentrates solutes and causes the phase separation of some polymer combinations.

An object of the present invention is to provide a stable pharmaceutical formulation comprising PEG-based prodrugs of ADM (PEG-ADM), which are delivered to the respiratory tract via inhalation.

Another object of the present invention is to provide suitable stable pharmaceutical formulations comprising PEG-based prodrugs of ADM (PEG-ADM) for treatment and/or prevention of ALI/ARDS, which are delivered to the respiratory tract via inhalation.

Moreover, it was an object of the present invention to allow the nebulization of aqueous formulations of PEG-ADM of therapeutically-relevant concentrations. Moreover, it was an object of the present invention to allow the nebulization of aqueous formulations of PEG-ADM of therapeutically-relevant concentrations by means of vibrating-mesh nebulizers.

Vibrating-mesh nebulizers are generally described in for example U.S. Pat. No. 6,467,476 B1, U.S. Pat. No. 8,398,001 B2 or U.S. Pat. No. 7,331,339 B2. Vibrating-mesh nebulizers comprise a thin plate, usually made from metal, the so-called mesh. The mesh comprises a front surface and a rear surface. The mesh has a plurality of apertures extending between the front surface and the rear surface. In some embodiments the apertures are tapered to narrow from the rear surface to the front surface. The liquid to be nebulized is usually in a reservoir in fluid communication with the rear surface of the mesh.

The efficiency of formulation nebulization (i.e. size of the generated aerosol particles and the output rate, whereby the output rate is defined as the mass of aerosol delivered by the nebulizer device per time) is on one hand a function of the aperture cross-section of the vibratory mesh of the employed vibrating-mesh nebulizer. On the other hand, the physicochemical properties of the utilized formulation also reveal significant impact on the delivery of aerosol particles from the nebulizer device. A number of studies investigated the interplay of formulation parameters with the mode of vibrating-mesh nebulization [Beck-Broichsitter M. and Oesterheld N., Eur J Pharm Biopharm, 119, 11-6 (2017)] in order to match the performance to the requirements of the individual application.

Micron-scale aperture dimensions are required for the generation of fine medicament mists suitable for inhalation to the deep lungs. However, the fabrication of apertures suitable for generating smallest particles is challenging [Kohn M. and Matsuoka Y., JSME Int J, Ser B 47(3), 497-500 (2004); Shen et al., Sens. Actuators A, 144(1), 135-43 (2008)]. Furthermore, despite sophisticated techniques being around to fabricate the aperture diameter of meshes to dimensions smaller than 5 μm, the variations in size between the apertures in a single mesh are still considerable due to the small overall dimensions. This will directly lead to significant differences of the efficiency of formulation nebulization from one vibrating-mesh nebulizer to another for the same pharmaceutical formulation.

One such example are aqueous formulations of PEG-ADM (i.e. a 40 kDa PEG conjugated to ADM; cf. compound according to formula (Ia) below) when nebulized by means of the Aerogen® Solo. PEG-ADM (see WO 2013/064508 A1) is described as compound which act as slow release ADM-prodrug with extended duration of pharmacological action which is intended for an application to self-breathing and ventilated patients. The Aerogen® Solo device is well-known to the person skilled in the art [El Hansy M., et al., Pulm Pharmacol Ther, 45(XX), 159-63 (2017); Dugernier J., et al., Ann Intensive Care, 6, 73 (2016); Ari A., et al., Respir Care 55(7), 837-44 (2010)]. A pharmaceutical formulation was developed for PEG-ADM. The pharmaceutical formulation comprises PEG-ADM (component a), a solvent (component b), a pH regulator (component c) and an osmolarity regulator (component d). The thermal characterization of the pharmaceutical formulation was investigated via Differential Scanning calorimetry (DSC). The results showed a relatively low glass transition temperature of −58° C. for the solutes in amorphous state, and a eutectic temperature of −22° C. for the solution with completely crystallized sodium chloride. These thermal properties clearly suggest that the solution is not stable unless it is present in fully frozen state at a storage temperature of approximately −58° C. ° C. or lower.

However, the alternative higher storage temperature of −20° C.+/−5° C. (equivalent to <−15° C.) was studied despite the negative thermal characteristics, and surprisingly sufficiently good stability of the labile PEG-ADM was observed in the applied analytical techniques. Thus, surprisingly, the stability results demonstrate that the pharmaceutical formulation can be stored at <−15° C. with good stability. At this temperature, the pharmaceutical formulation was partially frozen and partially liquid. Rather, the skilled person would have expected that only a completely frozen solution would have the required stability requirements. Thus, it was surprising that also the pharmaceutical formulation was only partially frozen, the stability was good. This elevated storage temperature constitutes a significant advantage with regard to supply chain, storage costs and availability of suitable storage capacities at the clinical centers. Such advantageous findings also apply to the pharmaceutical formulation and intermediates thereof (e.g. Examples 1 and 8 described in Sections B to D below). Moreover, the storage stability and the nebulization properties were investigated (see sections C and D below).

Surprisingly, it has been shown that the pharmaceutical formulation according to the invention has the following surprising technical effects
the pharmaceutical formulation is stable;
the pharmaceutical formulation is stable and shows good nebulization properties;
the pharmaceutical formulation is stable, even when frozen (frozen solution) or thawed;
the pharmaceutical formulation is still stable after freezing and/or thawing; this equally applies for re-thawed and re-frozen pharmaceutical formulations;
the pharmaceutical formulation shows after freezing and/or thawing still good nebulization properties;
the pharmaceutical formulation can be frozen and thawed several times without losing its stability and/or nebulization properties;
the pharmaceutical formulation can be stored below −15° C. with good stability—this elevated storage temperature constitutes a significant advantage with regard to supply chain, storage costs and availability of suitable storage capacities at the clinical centers.

Accordingly, the present invention provides a liquid pharmaceutical formulation comprising:
a. 0.04 mg/mL to 145 mg/mL of PEG-ADM, wherein the PEG-ADM is a compound according to the general formula (I),

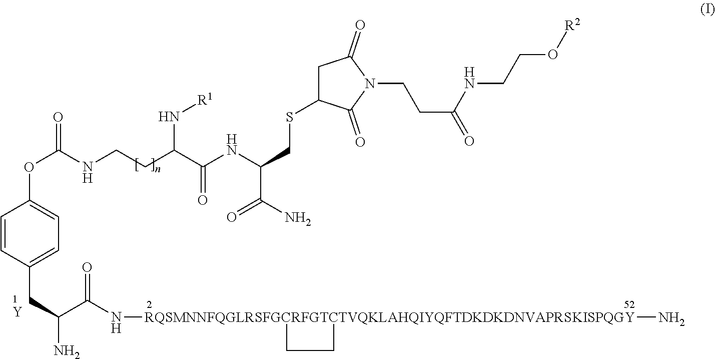

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
or a hydrate thereof, solvate thereof, salt thereof, pharmaceutically acceptable salt thereof, or the solvates of salts thereof;
b. a solvent;
c. a pH regulator; and
d. an osmolarity regulator;

wherein the pharmaceutical formulation has a pH of 3 to 5; and

wherein the osmolar concentration is between 150 to 450 mosmol/L, and

wherein the concentrations of the components are based on the total volume of the liquid pharmaceutical formulation.

The numbering of amino acids in formula (I) refers to the corresponding human adrenomedullin (ADM) sequence.

Pharmaceutical Formulation

The pharmaceutical formulation according to the invention is liquid. The term “pharmaceutical formulation” and “liquid pharmaceutical formulation” are synonyms. In one embodiment the pharmaceutical formulation according to the invention is for inhalation and/or inhalative use.

The pharmaceutical formulation according to the invention comprises components a, b, c and d. The components are described in detail below.

The concentrations of components are based on the total volume of the liquid pharmaceutical formulation. The pharmaceutical formulation has an osmotic concentration of 150 to 450 mosmol/L. The pharmaceutical formulation has a pH of 3 to 5.

Accordingly, even if not stated specifically in the embodiments of the invention disclosed herein, the following features apply to all embodiments disclosed:
when referring to “PEG-ADM” a compound according to formula (I), a hydrate thereof, solvate thereof, salt thereof, pharmaceutically acceptable salt thereof, or the solvates of salts thereof, are meant;
the concentrations of components a, b, c and d are based on the total volume of the liquid pharmaceutical formulation,
the pharmaceutical formulation has a pH of 3 to 5, and
pharmaceutical formulation has an osmotic concentration of 150 to 450 mosmol/L.

Some concentrations are given in “mg/mL”. Mass concentration of solution is expressed as “mg/mL” for “milligram per milliliter”. Here, a solid compound is dissolved in the liquid. For example, if 100 mg of sodium chloride is used to make up a total volume of 100 mL, then a 1 mg/mL solution of sodium chloride has been made. The concentrations of components are based on the total volume of the pharmaceutical formulation.

Moreover, the component c comprised in the pharmaceutical formulation can act also as osmolarity regulator (component d.) This means they can have overlapping functionality. For example, as described in more detail below, a buffer system of citric acid, sodium citrate and/or hydrochloric acid and sodium hydroxide would act as osmolarity regulator as well due to the ions contains in the solution. In that case the components c. and d. are present by one and the same component(s) d. Nevertheless, there are functionalities of the components c and d that overlap. However, these overlapping concentrations are disregarded when calculating the concentrations of the pH regulator or the osmolarity regulator, respectively. The osmolarity regulators are neutral salts, e.g. sodium chloride (NaCl). The pH regulators can contain salts or substances that contribute to osmolarity (e.g. buffer comprising citric acid, sodium citrate and hydrochloric acid comprises in solution sodium ions and chloride ions). The concentration of these contributing salts is not included in the concentration of the osmolarity regulator.

In one embodiment the pharmaceutical formulation according to the invention is a solution. The term “solution” is used as typically in the art. It refers to a homogeneous liquid preparation that contain one or more substances dissolved, i.e., molecularly dispersed, in a suitable solvent and/or mixture of mutually miscible solvents.

In one embodiment the pharmaceutical formulation according to the invention is a frozen solution. The term “frozen” means that the solution is at least partially frozen. In one embodiment, the pharmaceutical formulation is partially frozen.

In one embodiment the pharmaceutical formulation according to the invention is an aqueous solution. The aqueous solution substantially contains or consists of water as solvent b. “Substantially” here means greater than or equal to 80% by weight, 90% by weight, 95% by weight, 99% by weight or 99.9% by weight, in each case based on the total weight of the overall weight of the pharmaceutical formulation.

In one embodiment the liquid phase of the pharmaceutical formulation according to the invention substantially contains or consists of water. “Substantially” here means greater than or equal to 80% by weight, 90% by weight, 95% by weight, 96% by weight, 97% by weight, 98% by weight, 99% by weight or 99.9% by weight, in each case based on the total weight of the overall weight of the liquid phase.

In one embodiment the pharmaceutical formulation according to the invention is a dispersion. “Dispersions” and/or “disperse systems” are known in principle to a person skilled in the art (cf. “Pharmazeutische Technologie”, Voigt, Deutscher Apotheker Verlag Stuttgart, 2000, pp. 81 ff). Disperse phases can be classified according to their particle size as follows: molecularly dispersed solution having a particle size of <1 nm (e.g. real solution/fluid phases); colloidally dispersed dissolved having a particle size of greater and/or equal to 1 nm to 1 μm; and coarsely dispersed having a particle size of greater of 1 μm. In one embodiment the pharmaceutical formulation according to the present invention is an aqueous dispersion. The term “aqueous” is defined above and refers to the liquid phase of the dispersion.

PEG-ADM (Component a)

The pharmaceutical formulation according to the invention comprises PEG-ADM. The term “the compound of formula (I)” or “compound according to the general formula (I)” or “PEG-ADM” or “PEG-based prodrugs of ADM” or “component a” are used as synonyms and refer to a compound according to the general formula (I),

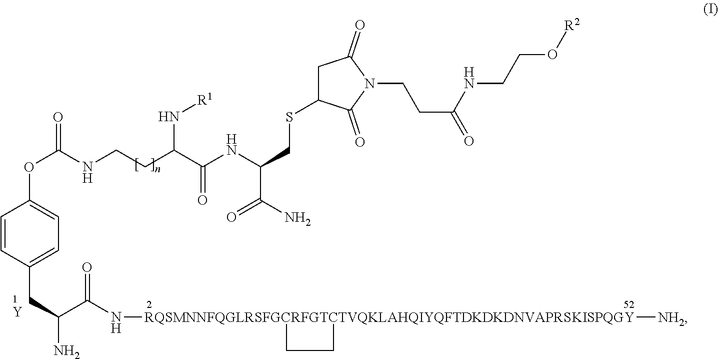

in which
   n represents the number 0, 1, 2 or 3,
   R¹ represents hydrogen, methyl, ethyl, n-propyl or isopropyl,
   R² represents linear or branched PEG 20 kDa to 80 kDa endcapped with a methoxy-group.

The term “PEG-ADM” also comprises a hydrate thereof, solvate thereof, salt thereof, pharmaceutically acceptable salt thereof, or the solvates of salts thereof. Thus, “PEG-ADM” is a synonym for the compounds according to formula (I), compounds according to formula (Ia), a hydrate thereof, solvate thereof, salt thereof, pharmaceutically acceptable salt thereof, or the solvates of salts thereof. The synthesis of PEG-ADM is described in WO 2013/064508 A1. PEG-ADM acts as a prodrug. In the body, adrenomedullin (ADM) is released from PEG-ADM. This is described in detail in WO 2013/064508 A1.

In one embodiment the pharmaceutical formulation the PEG-ADM is selected from compounds of the general formula (I),

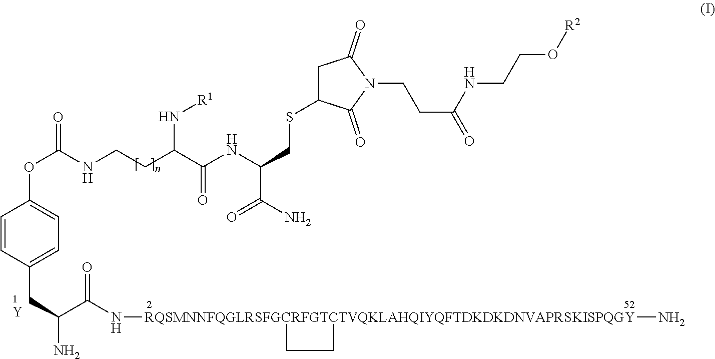

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group, a hydrate thereof, solvate thereof, salt thereof, pharmaceutically acceptable salt thereof, or the solvates of salts thereof.

In one embodiment the pharmaceutical formulation the PEG-ADM is selected from compounds of the formula (I) in which
n represents the number 1 or 2,
R¹ represents hydrogen or methyl,
R² represents linear PEG 40 kDa endcapped with a methoxy-group.

In one embodiment the pharmaceutical formulation the PEG-ADM is selected from compounds of the formula (I) in which
n represents the number 1 or 2,
R¹ represents hydrogen,
R² represents linear PEG 40 kDa endcapped with a methoxy-group.

In one embodiment the pharmaceutical formulation the PEG-ADM is the compound according to formula (Ia)

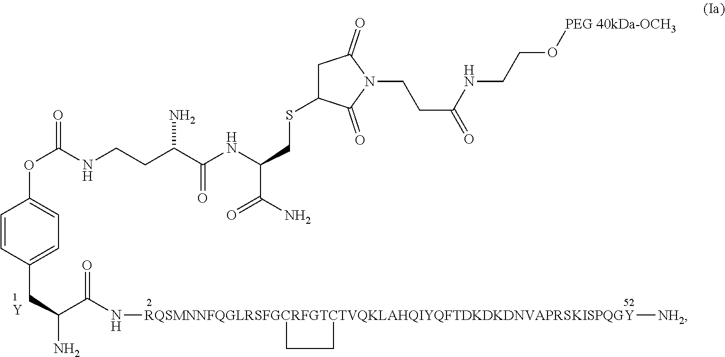

The compound according to formula (Ia) is described in detail in WO 2013/064508 A1. Its CAS number is 1432735-93-7.

In one embodiment of the pharmaceutical formulation according to the invention the PEG-ADM is the compound according to formula (Ia), a hydrate thereof, solvate thereof, salt thereof, pharmaceutically acceptable salt thereof, or the solvates of salts thereof.

Depending on their structure, the compounds according to the invention may exist in stereoisomeric forms (enantiomers, diastereomers). The invention therefore embraces the enantiomers or diastereomers and the particular mixtures thereof. The stereoisomerically homogeneous constituents can be isolated in a known manner from such mixtures of enantiomers and/or diastereomers.

When the compounds according to the invention can occur in tautomeric forms, the present invention embraces all tautomeric forms.

In the context of the present invention, preferred salts are physiologically acceptable salts of the compounds according to the invention.

“Physiologically acceptable salts” or “pharmaceutically acceptable salts” of the compounds according to the invention include acid addition salts of mineral acids, carboxylic acids and sulfonic acids, for example salts of hydrochloric acid, hydrobromic acid, sulfuric acid, phosphoric acid, methane sulfonic acid, ethane sulfonic acid, toluene sulfonic acid, benzenesulfonic acid, naphthalene disulfonic acid, acetic acid, trifluoroacetic acid, propionic acid, lactic acid, tartaric acid, maleic acid, citric acid, fumaric acid, maleic acid and benzoic acid.

“Physiologically acceptable salts” or “pharmaceutically acceptable salts” of the compounds according to the invention also include salts of customary bases, for example and with preference alkali metal salts (e.g. sodium and potassium salts), alkaline earth metal salts (e.g. calcium and magnesium salts) and ammonium salts derived from ammonia or organic amines having 1 to 16 carbon atoms, for example and with preference ethylamine, diethylamine, triethylamine, ethyl-diiso-propyl-amine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine. Suitable pharmaceutically acceptable salts that can be used in the combination according to the invention are well known to those skilled in the art and include salts of inorganic acids, organic acids, inorganic bases, alkaline cations, alkaline earth cations and organic bases. In one embodiment the pharmaceutically acceptable salt can be selected from hydrochloric acid, hydrobromic acid, sulfuric acid, phosphoric acid, methane sulphonic acid, trifluoromethanesulfonic acid, benzenesulfonic acid, p-toluene sulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid acetate, benzoate, besylate, bromide, camsylate, carbonate, citrate, edisylate, estolate, fumarate, gluceptate, gluconate, glucuronate, hippurate, iodide, isethionate, lactate, lactobionate, malate, maleate, mesylate, methylsulfate, napsylate, nitrate, oxalate, pamoate, phosphate, stearate, succinate, sulfate, tartrate, bitartrate, tosylate, calcium, diolamine, lithium, lysine, magnesium, meglumine, N-methylglucamine, oleamine, potassium, tromethamine, tris(hydroxymethyl)aminomethane, benzenesulfonate, ethanesulfonate and zinc.

In one embodiment the pharmaceutically acceptable salt can be selected from hydrochloride, sulfate, mesylate, tosylate, tartrate, citrate, benzenesulfonate, ethanesulfonate, maleate, and phosphate

In the context of the invention, solvates refer to those forms of the compounds according to the invention which, in the solid or liquid state, form a complex by coordination with solvent molecules. Hydrates are a specific form of the solvates, in which the coordination is with water. Preferred solvates in the context of the present invention are hydrates.

The pharmaceutical formulation according to the invention comprises 0.04 mg/mL to 145 mg/mL of PEG-ADM. The concentration of component a is based on the total volume of the liquid pharmaceutical formulation.

As explained above, PEG-ADM acts as a prodrug. ADM is released from PEG-ADM (cf. WO 2013/064508 A1). In therapy, the amount of ADM comprised in a medicament and/or the ADM released from the prodrug PEG-ADM in the body is an important aspect. Moreover, the respective concentration or amount of ADM comprised in a certain amount of PEG-ADM can widely vary depending on the length of the PEG chain. The length of the PEG chain has an impact on the weight of the PEG-ADM, and, thus, on the amount of PEG-ADM that is needed to provide for a certain concentration of ADM. For example, for a PEG-ADM according to formula (I), wherein R2 comprises a PEG 20 kDa endcapped with a methoxy-group, approximately 1 mg ADM is comprised in approximately 4.4 mg PEG-ADM. For example, for a PEG-ADM according to formula (I), wherein R2 represents linear PEG 40 kDa endcapped with a methoxy-group (cf. compound according to formula (Ia)), approximately 1 mg ADM is comprised in approximately 7.7 mg PEG-ADM. For example, for a PEG-ADM according to formula (I), wherein R2 comprises a PEG 80 kDa endcapped with a methoxy-group, approximately 1 mg ADM is comprised in approximately 14.35 mg PEG-ADM. Therefore, the concentrations given for PEG-ADM herein are approximations.

In one embodiment the pharmaceutical formulation according to the invention comprises 0.077 mg/mL to 77 mg/mL of PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.85 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 7.7 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 2.31 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.85 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 7.7 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 38.5 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 38.5 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 21.3 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 2.31 mg/mL to 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 2.31 mg/mL to 3.85 mg/mL PEG-ADM, wherein the PEG-ADM is a compound according to the general formula (I) as defined in any one of the embodiments disclosed for PEG-ADM herein, a hydrate thereof, solvate thereof, salt thereof pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 23.1 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 23.1 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 6.16 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 4.62 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.85 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 00.37 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.696 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the pharmaceutical formulation according to the invention comprises 2.31 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment, the pharmaceutical formulation comprises approximately 0.044 mg/mL to 44 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the pharmaceutical formulation comprises approximately 0.22 mg/mL to 22 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the pharmaceutical formulation comprises approximately 0.44 mg/mL to 13.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the pharmaceutical formulation comprises approximately 0.44 mg/mL to 4.4 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the pharmaceutical formulation comprises approximately 1.3 mg/mL to 2.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the pharmaceutical formulation comprises approximately 0.14 mg/mL to 144 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the pharmaceutical formulation comprises approximately 0.7 mg/mL to 71.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the pharmaceutical formulation comprises approximately 1.4 mg/mL to 43 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the pharmaceutical formulation comprises approximately 1.4 mg/mL to 14.3 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the pharmaceutical formulation comprises approximately 4.3 mg/mL to 7.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment the pharmaceutical formulation according to the invention comprises 0.04 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 3.08 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 1.54 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 0.77 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 0.385 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.04 mg/mL to 0.2 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 3.08 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 1.54 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 0.77 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.2 mg/mL to 0.385 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 3.08 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 1.54 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.385 mg/mL to 0.77 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 3.08 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 0.77 mg/mL to 1.54 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 1.54 mg/mL to 3.08 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.08 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.696 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.696 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.696 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.696 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 3.696 mg/mL to 4.62 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 4.62 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 4.62 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 4.62 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 4.62 mg/mL to 6.16 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 6.16 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 6.16 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 6.16 mg/mL to 7.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 7.7 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 7.7 mg/mL to 10 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises 10 mg/mL to 23.1 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the pharmaceutical formulation according to the invention comprises a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.385 mg/mL, 0.77 mg/mL, 1.54 mg/mL, 3.08 mg/mL, 3.696 mg/mL, 4.62 mg/mL, 6.16 mg/mL and 7.7 mg/mL.

In one embodiment the pharmaceutical formulation according to the invention comprises a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.04 mg/mL, 0.02 mg/mL, 0.385 mg/mL, 0.77 mg/mL, 1.54 mg/mL, 3.08 mg/mL, 3.696 mg/mL, 4.62 mg/mL, 6.16 mg/mL, 7.7 mg/mL, 10 mg/mL and 23.1 mg/mL.

In one embodiment the pharmaceutical formulation according to the invention comprises a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.02 mg/mL, 0.385 mg/mL, 0.77 mg/mL, 1.54 mg/mL, 3.08 mg/mL, 3.696 mg/mL, 4.62 mg/mL, 6.16 mg/mL, 7.7 mg/mL, 10 mg/mL and 23.1 mg/mL.

In one embodiment the pharmaceutical formulation according to the invention comprises a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.02 mg/mL, 0.385 mg/mL, 0.77 mg/mL, 1.54 mg/mL, 3.08 mg/mL, 3.696 mg/mL, 4.62 mg/mL, 6.16 mg/mL, 7.7 mg/mL and 10 mg/mL.

In one embodiment the pharmaceutical formulation according to the invention comprises a PEG-ADM according to formula (I) or formula (Ia), wherein the ADM concentration comprised in the PEG-ADM is selected from

0.026 mg/mL to 0.05 mg/mL;
0.026 mg/mL to 0.1 mg/mL;
0.026 mg/mL to 0.2 mg/mL;
0.026 mg/mL to 0.4 mg/mL;
0.026 mg/mL to 0.48 mg/mL;
0.026 mg/mL to 0.6 mg/mL;
0.026 mg/mL to 0.8 mg/mL;
0.026 mg/mL to 1 mg/mL;
0.026 mg/mL to 1.3 mg/mL;
0.026 mg/mL to 3 mg/mL;
0.05 mg/mL to 0.1 mg/mL;
0.05 mg/mL to 0.2 mg/mL;
0.05 mg/mL to 0.4 mg/mL;
0.05 mg/mL to 0.48 mg/mL;
0.05 mg/mL to 0.6 mg/mL;
0.05 mg/mL to 0.8 mg/mL;
0.05 mg/mL to 1 mg/mL;
0.05 mg/mL to 1.3 mg/mL;
0.05 mg/mL to 3 mg/mL;
0.1 mg/mL to 0.2 mg/mL;
0.1 mg/mL to 0.4 mg/mL;
0.1 mg/mL to 0.48 mg/mL;
0.1 mg/mL to 0.6 mg/mL;
0.1 mg/mL to 0.8 mg/mL;
0.1 mg/mL to 1 mg/mL;
0.1 mg/mL to 1.3 mg/mL;
0.1 mg/mL to 3 mg/mL;
0.2 mg/mL to 0.4 mg/mL;
0.2 mg/mL to 0.48 mg/mL;
0.2 mg/mL to 0.6 mg/mL;
0.2 mg/mL to 0.8 mg/mL;
0.2 mg/mL to 1 mg/mL;
0.2 mg/mL to 1.3 mg/mL;
0.2 mg/mL to 3 mg/mL;
0.4 mg/mL to 0.48 mg/mL;
0.4 mg/mL to 0.6 mg/mL;
0.4 mg/mL to 0.8 mg/mL;
0.4 mg/mL to 1 mg/mL;
0.4 mg/mL to 1.3 mg/mL;
0.4 mg/mL to 3 mg/mL;
0.48 mg/mL to 0.6 mg/mL;
0.48 mg/mL to 0.8 mg/mL;
0.48 mg/mL to 1 mg/mL;
0.48 mg/mL to 1.3 mg/mL;
0.48 mg/mL to 3 mg/mL;
0.6 mg/mL to 0.8 mg/mL;
0.6 mg/mL to 1 mg/mL;
0.6 mg/mL to 1.3 mg/mL;
0.6 mg/mL to 3 mg/mL;
0.8 mg/mL to 1 mg/mL;
0.8 mg/mL to 1.3 mg/mL;
0.8 mg/mL to 3 mg/mL;
1.3 mg/mL to 1.3 mg/mL; and
1.3 mg/mL to 3 mg/mL.

Solvent (Component b)

The pharmaceutical formulation according to the invention comprises a solvent. The term “solvent” is used as typically in the art. The terms “solvent” and “component b” are synonyms. The teen solvent refers to pure solvents and/or to mixtures of different solvents.

In one embodiment of the pharmaceutical formulation according to the invention, the solvent comprises water. In one embodiment of the pharmaceutical formulation according to the invention, the solvent consists of water.

pH Regulator (Component c)

The pharmaceutical formulation according to the invention comprises a pH regulator. The term “pH regulator” and “component c” are synonyms. The term “pH regulator” comprises substances that regulate the pH. The term “pH regulator” also refers to a plurality of pH regulators. The term “pH regulator” refers to one pH regulator or two or more pH regulators. Thus, the term “pH regulator” also encompasses mixtures comprising or consisting of different pH regulators. When a plurality of pH regulators is given, the sum of the concentrations of these pH regulators are the total concentration of the pH regulator. For example, if a concentration of 1 mg/ml citric acid and 1 mg/ml sodium hydroxide is given, the total concentration is 2 mg/mL pH regulator.

One example of a pH regulator is a buffer system. A “buffer” consists of a mixture of a weak acid and its conjugate base, or vice versa. Its pH changes very little when a small amount of strong acid or base is added to it. Buffer solutions are used as a means of keeping pH at a nearly constant value in a wide variety of chemical applications. One example is the system citrate/citric acid. The citrate is the salt of citric acid, e.g. the sodium salt, the potassium salt or the calcium salt of citric acid. Further examples of salts, pharmaceutical acceptable salts, derivatives of citric acid encompass citric acid anhydrous, sodium citrate and citric acid monohydrate. Embodiments of buffers that can be used in the formulations according to the invention are phosphate citrate buffer (pH 2.2-8.0, pKa=7.2/6.4), citrate buffer (pH 3-6.2; pKa 6.15-8.06), sodium acetate buffer (pH 3.6-5.6, pKa 4.76), glycine-HCl (pH 2.2-3.6, pKa 2.35). Even if not explicitly stated herein, any buffer that is suitable for adjusting the pH to 3 to 5 can be used in the pharmaceutical formulation according to the invention.

In one embodiment the pH regulator comprises citric acid, a salt of citric acid, a pharmaceutical acceptable salt of citric acid, a derivative of citric acid, and/or mixtures thereof.

In one embodiment the pH regulator comprises hydrochloric acid, citric acid, a salt of citric acid, pharmaceutical acceptable salt of citric acid, derivative of citric acid, and/or mixtures thereof.

In one embodiment, the pH regulator comprises hydrochloric acid.

In one embodiment, the pH regulator comprises a mixture comprising hydrochloric acid and sodium hydroxide. In one embodiment, the pH regulator comprises a mixture comprising hydrochloric acid, sodium hydroxide and citric acid. In one embodiment, the pH regulator comprises a mixture comprising sodium citrate and hydrochloric acid. In an alternative of these embodiments listed before, the citric acid is a salt of citric acid, pharmaceutical acceptable salt of citric acid, a derivative of citric acid and/or mixtures thereof, preferably citric acid anhydrous, sodium citrate and citric acid monohydrate.

In one embodiment, the pH regulator consists of hydrochloric acid. In one embodiment, the pH regulator consists of a mixture comprising hydrochloric acid and sodium hydroxide. In one embodiment, the pH regulator consists of a mixture comprising hydrochloric acid, sodium hydroxide and citric acid. In one embodiment, the pH regulator consists of a mixture comprising sodium hydroxide and citric acid. In one embodiment, the pH regulator consists of a mixture comprising sodium citrate and hydrochloric acid. In an alternative of the embodiments listed before, the citric acid is a salt of citric acid, pharmaceutical acceptable salt of citric acid, a derivative of citric acid and/or mixtures thereof, preferably citric acid anhydrous, sodium citrate and citric acid monohydrate.

In one embodiment the pharmaceutical formulation according to the invention comprises at least one pH regulator. In one embodiment the pharmaceutical formulation according to the invention comprises two or more pH regulators. In one embodiment the pharmaceutical formulation according to the invention comprises three or more pH regulators. In one embodiment the pharmaceutical formulation according to the invention comprises mixtures of pH regulators.

In one embodiment, the pharmaceutical formulation comprises 0.1 mg/mL to 250 mg/mL of the pH regulator. In one embodiment, the pharmaceutical formulation comprises 0.3 mg/mL to 250 mg/mL of the pH regulator. In one embodiment, the pharmaceutical formulation comprises 0.5 mg/mL to 100 mg/mL of the pH regulator. In one embodiment, the pharmaceutical formulation comprises 0.9 mg/mL to 90 mg/mL of the pH regulator. In one embodiment, the pharmaceutical formulation comprises 2.5 mg/mL to 46 mg/mL of the pH regulator. In one embodiment, the pharmaceutical formulation comprises 7.8 mg/mL to 29 mg/mL of the pH regulator. In one embodiment, the pharmaceutical formulation comprises 12.5 mg/mL to 19 mg/mL of the pH regulator. In one embodiment, the pharmaceutical formulation comprises 0.01 mg/mL to 100 mg/mL of the pH regulator. In one embodiment, the pharmaceutical formulation comprises 0.1 mg/mL to 50 mg/mL of the pH regulator. In one embodiment, the pharmaceutical formulation comprises 0.5 mg/mL to 25 mg/mL of the pH regulator. In one embodiment, the pharmaceutical formulation comprises 0.8 mg/mL to 15 mg/mL of the pH regulator. In one embodiment, the pharmaceutical formulation comprises 1.5 mg/mL to 9 mg/mL of the pH regulator.

The concentration of component c. is based on the total volume of the liquid pharmaceutical formulation.

In one embodiment the pharmaceutical formulation comprises 0.1 mg/mL to 100 mg/mL citric acid, a salt of citric acid, pharmaceutical acceptable salt of citric acid, a derivative of citric acid and/or mixtures thereof. In one embodiment the pharmaceutical formulation comprises 0.3 mg/mL to 30 mg/mL citric acid, a salt of citric acid, pharmaceutical acceptable salt of citric acid, a derivative of citric acid and/or mixtures thereof. In one embodiment the pharmaceutical formulation comprises 1 mg/mL to 15 mg/mL citric acid, a salt of citric acid, pharmaceutical acceptable salt of citric acid, a derivative of citric acid and/or mixtures thereof. In one embodiment the pharmaceutical formulation comprises 2 mg/mL to 10 mg/mL citric acid, a salt of citric acid, pharmaceutical acceptable salt of citric acid, a derivative of citric acid and/or mixtures thereof. In one embodiment the pharmaceutical formulation comprises 4 mg/mL to 7 mg/mL citric acid, a salt of citric acid, pharmaceutical acceptable salt of citric acid, a derivative of citric acid and/or mixtures thereof. In an alternative of the embodiments listed before, the salt of citric acid, pharmaceutical acceptable salt of citric acid, a derivative of citric acid and/or mixtures thereof, are selected from citric acid anhydrous, sodium citrate and citric acid monohydrate.

In one embodiment the pharmaceutical formulation comprises 0.01 mg/mL to 50 mg/mL sodium hydroxide. In one embodiment the pharmaceutical formulation comprises 0.1 mg/mL to 10 mg/mL sodium hydroxide. In one embodiment the pharmaceutical formulation comprises 0.5 mg/mL to 6 mg/mL sodium hydroxide. In one embodiment the pharmaceutical formulation comprises 0.8 mg/mL to 4 mg/mL sodium hydroxide. In one embodiment the pharmaceutical formulation comprises 1.5 mg/mL to 3 mg/mL sodium hydroxide.

In one embodiment the pharmaceutical formulation comprises 0.1 mg/mL to 100 mg/mL hydrochloric acid. In one embodiment the pharmaceutical formulation comprises 0.5 mg/mL to 50 mg/mL hydrochloric acid. In one embodiment the pharmaceutical formulation comprises 1 mg/mL to 25 mg/mL hydrochloric acid. In one embodiment the pharmaceutical formulation comprises 5 mg/mL to 15 mg/mL hydrochloric acid.

In one embodiment the pharmaceutical formulation comprises 7 mg/mL to 9 mg/mL hydrochloric acid. In one alternative of these embodiments, the hydrochloric acid is or comprises hydrochloric acid 10% (m/V).

In one embodiment, the pharmaceutical formulation comprises as component c the following mixture of pH regulators
0.1 mg/mL to 100 mg/mL citric acid, a salt of citric acid, pharmaceutical acceptable salt of citric acid, a derivative of citric acid and/or mixtures thereof;
0.01 mg/mL to 50 mg/mL sodium hydroxide; and
0.1 mg/mL to 100 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises as component b the following mixture of pH regulators
0.3 mg/mL to 30 mg/mL citric acid, a salt of citric acid, pharmaceutical acceptable salt of citric acid, a derivative of citric acid and/or mixtures thereof;
0.1 mg/mL to 10 mg/mL sodium hydroxide; and
0.5 mg/mL to 50 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises as component b the following mixture of pH regulators
1 mg/mL to 15 mg/mL citric acid, a salt of citric acid, pharmaceutical acceptable salt of citric acid, a derivative of citric acid and/or mixtures thereof;
0.5 mg/mL to 6 mg/mL sodium hydroxide; and
1 mg/mL to 25 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises as component b the following mixture of pH regulators
2 mg/mL to 10 mg/mL citric acid, a salt of citric acid, pharmaceutical acceptable salt of citric acid, a derivative of citric acid and/or mixtures thereof;
0.8 mg/mL to 4 mg/mL sodium hydroxide; and
5 mg/mL to 15 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises as component b the following mixture of pH regulators
4 mg/mL to 7 mg/mL citric acid, a salt of citric acid, pharmaceutical acceptable salt of citric acid, a derivative of citric acid and/or mixtures thereof;
1.5 mg/mL to 3 mg/mL sodium hydroxide; and
7 mg/mL to 9 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.077 mg/mL to 77 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
0.1 mg/mL to 100 mg/mL citric acid;
0.01 mg/mL to 50 mg/mL sodium hydroxide;
0.1 mg/mL to 100 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.385 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
0.3 mg/mL to 30 mg/mL citric acid;
0.1 mg/mL to 10 mg/mL sodium hydroxide;
0.5 mg/mL to 50 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.77 mg/mL to 23.1 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
1 mg/mL to 15 mg/mL citric acid;
0.5 mg/mL to 6 mg/mL sodium hydroxide;
1 mg/mL to 25 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.77 mg/mL to 7.7 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
2 mg/mL to 10 mg/mL citric acid;
0.8 mg/mL to 4 mg/mL sodium hydroxide; and
5 mg/mL to 15 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
2.31 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
4 mg/mL to 7 mg/mL citric acid;
1.5 mg/mL to 3 mg/mL sodium hydroxide; and
7 mg/mL to 9 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.077 mg/mL to 77 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
0.1 mg/mL to 100 mg/mL citric acid;
0.01 mg/mL to 50 mg/mL sodium hydroxide;
0.1 mg/mL to 100 mg/mL hydrochloric acid;

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.385 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
0.3 mg/mL to 30 mg/mL citric acid;
0.1 mg/mL to 10 mg/mL sodium hydroxide;
0.5 mg/mL to 50 mg/mL hydrochloric acid;

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.77 mg/mL to 23.1 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
1 mg/mL to 15 mg/mL citric acid;
0.5 mg/mL to 6 mg/mL sodium hydroxide;
1 mg/mL to 25 mg/mL hydrochloric acid;

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
0.77 mg/mL to 7.7 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, 2 mg/mL to 10 mg/mL citric acid;
0.8 mg/mL to 4 mg/mL sodium hydroxide;
5 mg/mL to 15 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the pharmaceutical formulation comprises
2.31 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
4 mg/mL to 7 mg/mL citric acid;
1.5 mg/mL to 3 mg/mL sodium hydroxide;
7 mg/mL to 9 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

pH of the Pharmaceutical Formulation

The pharmaceutical formulation according to the invention has a pH of 3 to 5. In one embodiment the pharmaceutical formulation according to the invention formulation has a pH of 3.5 to 4.5. In one embodiment the pharmaceutical formulation according to the invention has a pH of 3 to 4. In one embodiment the pharmaceutical formulation according to the invention has a pH of 3 to 3.5. In one embodiment the pharmaceutical formulation according to the invention has a pH of 3.25 to 3.75. In one embodiment the pharmaceutical formulation according to the invention has a pH of 3.5 to 4. In one embodiment the pharmaceutical formulation according to the invention has a pH of 3 In one embodiment the pharmaceutical formulation according to the invention has a pH of 3.5. In one embodiment the pharmaceutical formulation according to the invention has a pH of 4. In one embodiment the pharmaceutical formulation according to the invention has a pH of 4 In one embodiment the pharmaceutical formulation according to the invention has a pH of 5.

Osmolarity Regulator (Component d)

The pharmaceutical formulation according to the invention comprises an osmolarity regulator. The term “osmolarity regulator” and “component d” are synonyms. The term “osmolarity regulator” refers to one osmolarity regulator as well as to mixtures of one two or more compounds for adjusting osmolarity. The osmotic concentration was determined via freezing-point depression [Osmomat 030, Gonotec, Model 030-D3P]. When a plurality of osmolarity regulators is given, the sum of the concentrations of these osmolarity regulators are the total concentration of the pH regulator. For example, if a concentration of 1 mg/ml sodium chloride and 1 mg/ml citric is given, the total concentration is 2 mg/mL osmolarity regulators.

In one embodiment of the pharmaceutical formulation according to the invention the osmolarity regulator is sodium chloride, citric acid, a salt, pharmaceutical acceptable salt, derivative of citric acid and/or mixtures thereof.

In one embodiment of the pharmaceutical formulation according to the invention the osmolarity regulator is citric acid, a salt, pharmaceutical acceptable salt, derivative of citric acid. In one embodiment of the pharmaceutical formulation according to the invention the osmolarity regulator is a salt, pharmaceutical acceptable salt, derivative of citric acid selected from the group consisting of citric acid anhydrous, sodium citrate and citric acid monohydrate.

In one embodiment of the pharmaceutical formulation according to the invention the osmolarity regulator is sodium chloride.

In one embodiment the pharmaceutical formulation according to the invention comprises 0.01 mg/mL to 100 mg/mL of an osmolarity regulator. The concentration of component d. is based on the total volume of the liquid pharmaceutical formulation.

In one embodiment the pharmaceutical formulation according to the invention the pharmaceutical formulation comprises 0.1 mg/mL to 30 mg/mL of the osmolarity regulator. In one embodiment the pharmaceutical formulation according to the invention the pharmaceutical formulation comprises 0.5 mg/mL to 15 mg/mL of the osmolarity regulator. In one embodiment the pharmaceutical formulation according to the invention the pharmaceutical formulation comprises 2 mg/mL to 10 mg/mL of the osmolarity regulator. In one embodiment the pharmaceutical formulation according to the invention the pharmaceutical formulation comprises 5 mg/mL to 7 mg/mL of the osmolarity regulator. In these embodiments listed before, the osmolarity regulator can be any embodiment disclosed herein for component d.

Osmolar Concentration of the Pharmaceutical Formulation

In the pharmaceutical formulation according to the invention, the osmolar concentration is between 150 to 450 mosmol/L. The osmolarity is expressed as osmotic concentration of “mosmol/l” or “milliosmole per liter”. In one embodiment the pharmaceutical formulation has an osmotic concentration between 150 to 450 mosmol/l. In one embodiment the pharmaceutical formulation has an osmotic concentration between 200 to 400 mosmol/l. In one embodiment the pharmaceutical formulation has an osmotic concentration between 270 to 330 mosmol/l. In one embodiment the pharmaceutical formulation has an osmotic concentration between 250 to 310 mosmol/l. In one embodiment the pharmaceutical formulation has an osmotic concentration of 300 mosmol/l.

Viscosity of the Pharmaceutical Formulation

The pharmaceutical formulation according to the invention ca also be characterized by its viscosity. The unit for viscosity is “millipascal second” or “mPa*s”. The viscosity was determined by an automatic rolling ball viscometer method according to Ph.Eur. 2.2.49 (2018), using an Anton Paar AMVn Automated Microviscometer

In one embodiment the viscosity of the formulation according to the invention is 0.9 to 2.2 mPa*s. In one embodiment the viscosity of the formulation according to the invention is approximately 1 to 2 mPa*s. In one embodiment the viscosity of the formulation according to the invention is approximately 1.05 to 2 mPa*s. In one embodiment the viscosity of the formulation according to the invention is approximately 1.05 to 1.9 mPa*s. In one embodiment the viscosity of the formulation according to the invention is approximately 1.1 to 2 mPa*s. In one embodiment the viscosity of the formulation according to the invention is approximately 1.05 mPa*s. In one embodiment the viscosity of the formulation according to the invention is approximately 1.1 mPa*s. In one embodiment the viscosity of the formulation according to the invention is approximately 1.2 mPa*s. In one embodiment the viscosity of the formulation according to the invention is approximately 1.3 mPa*s. In one embodiment the viscosity of the formulation according to the invention is approximately 1.4 mPa*s. In one embodiment the viscosity of the formulation according to the invention is approximately 1.5 mPa*s. In one embodiment the viscosity of the formulation according to the invention is approximately 1.9 mPa*s. In one embodiment the viscosity of the formulation according to the invention is approximately 2 mPa*s.

### Further Embodiments of the Pharmaceutical Formulation

In one embodiment the pharmaceutical formulation comprises
0.077 mg/mL to 77 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
0.1 mg/mL to 100 mg/mL citric acid;
0.01 mg/mL to 50 mg/mL sodium hydroxide;
0.1 mg/mL to 100 mg/mL hydrochloric acid;
0.01 mg/mL to 100 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the pharmaceutical formulation comprises
0.385 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
0.3 mg/mL to 30 mg/mL citric acid;
0.1 mg/mL to 10 mg/mL sodium hydroxide;
0.5 mg/mL to 50 mg/mL hydrochloric acid;
0.1 mg/mL to 30 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the pharmaceutical formulation comprises
0.77 mg/mL to 23.1 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
1 mg/mL to 15 mg/mL citric acid;
0.5 mg/mL to 6 mg/mL sodium hydroxide;
1 mg/mL to 25 mg/mL hydrochloric acid;
0.5 mg/mL to 15 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the pharmaceutical formulation comprises
0.77 mg/mL to 7.7 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
2 mg/mL to 10 mg/mL citric acid;
0.8 mg/mL to 4 mg/mL sodium hydroxide;
5 mg/mL to 15 mg/mL hydrochloric acid;
2 mg/mL to 10 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the pharmaceutical formulation comprises
2.31 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof,
4 mg/mL to 7 mg/mL citric acid;
1.5 mg/mL to 3 mg/mL sodium hydroxide;
7 mg/mL to 9 mg/mL hydrochloric acid;
5 mg/mL to 7 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the pharmaceutical formulation according to the invention the pharmaceutical formulation comprises
0.01 mg/mL to 10 mg/mL of PEG-ADM, wherein the PEG-ADM is a compound according to formula (I) or (Ia), wherein the concentration refers to the ADM concentration comprised in the PEG-ADM;
solvent;
0.1 mg/mL to 100 mg/mL of citric acid,
0.01 mg/mL to 50 mg/mL of sodium hydroxide,
0.1 mg/mL to 100 mg/mL hydrochloric acid 10% (m/V); and
0.01 mg/mL to 100 mg/mL of sodium chloride,

wherein the concentrations of the components are based on the total volume of the liquid pharmaceutical formulation; wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the pharmaceutical formulation according to the invention the pharmaceutical formulation comprises
0.01 mg/mL to 10 mg/mL of PEG-ADM, wherein the PEG-ADM is a compound according to formula (Ia), wherein the concentration refers to the ADM concentration comprised in the PEG-ADM;
water;
0.1 mg/mL to 100 mg/mL of citric acid,
0.01 mg/mL to 50 mg/mL of sodium hydroxide,
0.1 mg/mL to 100 mg/mL hydrochloric acid 10% (m/V); and
0.01 mg/mL to 100 mg/mL of sodium chloride,

wherein the concentrations of the components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the pharmaceutical formulation according to the invention the pharmaceutical formulation comprises
0.05 mg/mL to 5 mg/mL of PEG-ADM, wherein the PEG-ADM is a compound according to formula (I) or (Ia), wherein the concentration refers to the ADM concentration comprised in the PEG-ADM;
solvent;
0.3 mg/mL to 30 mg/mL of citric acid,
0.1 mg/mL to 10 mg/mL of sodium hydroxide,
0.5 mg/mL to 50 mg/mL hydrochloric acid; and
0.1 mg/mL to 30 mg/mL of sodium chloride,

wherein the concentrations of the components are based on the total volume of the liquid pharmaceutical formulation; wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the pharmaceutical formulation according to the invention the pharmaceutical formulation comprises
0.05 mg/mL to 5 mg/mL of PEG-ADM, wherein the PEG-ADM is a compound according to formula (Ia), wherein the concentration refers to the ADM concentration comprised in the PEG-ADM;
water;
0.3 mg/mL to 30 mg/mL of citric acid,
0.1 mg/mL to 10 mg/mL of sodium hydroxide,
0.5 mg/mL to 50 mg/mL hydrochloric acid 10% (m/V); and
0.1 mg/mL to 30 mg/mL of sodium chloride,

wherein the concentrations of the components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the pharmaceutical formulation according to the invention the pharmaceutical formulation comprises
0.1 mg/mL to 3 mg/mL of PEG-ADM, wherein the PEG-ADM is a compound according to formula (I) or (Ia), wherein the concentration refers to the ADM concentration comprised in the PEG-ADM;
solvent;
1 mg/mL to 15 mg/mL of citric acid,
0.5 mg/mL to 6 mg/mL of sodium hydroxide,
1 mg/mL to 25 mg/mL hydrochloric acid and
0.5 mg/mL to 15 of sodium chloride,

wherein the concentrations of the components are based on the total volume of the liquid pharmaceutical formulation;

wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (N); wherein optionally the solvent is or comprises water.

In one embodiment the pharmaceutical formulation according to the invention the pharmaceutical formulation comprises
0.1 mg/mL to 3 mg/mL of PEG-ADM, wherein the PEG-ADM is a compound according to formula (Ia), wherein the concentration refers to the ADM concentration comprised in the PEG-ADM;
water;
1 mg/mL to 15 mg/mL of citric acid,
0.5 mg/mL to 6 mg/mL of sodium hydroxide,
1 mg/mL to 25 mg/mL hydrochloric acid 10% (m/V); and
0.5 mg/mL to 15 mg/mL of sodium chloride,

wherein the concentrations of the components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the pharmaceutical formulation according to the invention the pharmaceutical formulation comprises
0.1 mg/mL to 1 mg/mL of PEG-ADM, wherein the PEG-ADM is a compound according to formula (I) or (Ia), wherein the concentration refers to the ADM concentration comprised in the PEG-ADM;
   solvent;
2 mg/mL to 10 mg/mL of citric acid,
0.8 mg/mL to 4 mg/mL of sodium hydroxide,
5 mg/mL to 15 mg/mL hydrochloric acid and
2 mg/mL to 10 mg/mL of sodium chloride,

wherein the concentrations of the components are based on the total volume of the liquid pharmaceutical formulation; wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the pharmaceutical formulation according to the invention the pharmaceutical formulation comprises
0.1 mg/mL to 1 mg/mL of PEG-ADM, wherein the PEG-ADM is a compound according to formula (Ia), wherein the concentration refers to the ADM concentration comprised in the PEG-ADM;
water;
2 mg/mL to 10 mg/mL of citric acid,
0.8 mg/mL to 4 mg/mL of sodium hydroxide,
5 mg/mL to 15 mg/mL hydrochloric acid 10% (m/V); and
2 mg/mL to 10 mg/mL of sodium chloride,

wherein the concentrations of the components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the pharmaceutical formulation according to the invention the pharmaceutical formulation comprises
0.3 mg/mL to 0.5 mg/mL of PEG-ADM, wherein the PEG-ADM is a compound according to formula (I) or (Ia), wherein the concentration refers to the ADM concentration comprised in the PEG-ADM; solvent;
4 mg/mL to 7 mg/mL of citric acid,
1.5 mg/mL to 3 mg/mL of sodium hydroxide,
7 mg/mL to 9 mg/mL hydrochloric acid and
5 mg/mL to 7 mg/mL of sodium chloride,

wherein the concentrations of the components are based on the total volume of the liquid pharmaceutical formulation; wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the pharmaceutical formulation according to the invention the pharmaceutical formulation comprises
0.3 mg/mL to 0.5 mg/mL of PEG-ADM, wherein the PEG-ADM is a compound according to formula (Ia), wherein the concentration refers to the ADM concentration comprised in the PEG-ADM;
water
4 mg/mL to 7 mg/mL of citric acid,
1.5 mg/mL to 3 mg/mL of sodium hydroxide,
7 mg/mL to 9 mg/mL hydrochloric acid 10% (m/V); and
5 mg/mL to 7 mg/mL of sodium chloride,

wherein the concentrations of the components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the pharmaceutical formulation according to the invention the pharmaceutical formulation comprises
0.48 mg/mL of PEG-ADM, wherein the PEG-ADM is a compound according to formula (Ia), wherein the concentration refers to the ADM concentration comprised in the PEG-ADM;
water;
5.38 mg/mL of citric acid anhydrous,
2.24 mg/mL of sodium hydroxide,
8.07 mg/mL hydrochloric acid 10% (m/V); and
6.54 mg/mL of sodium chloride,

wherein the concentrations of the components are based on the total volume of the liquid pharmaceutical formulation; wherein the aqueous formulation has a pH of 3.5 to 4.5, preferably a pH 3 to 4, more preferably a pH of 4.

In one embodiment the pharmaceutical formulation according to the invention the pharmaceutical formulation comprises
1 mg/mL of PEG-ADM, wherein the PEG-ADM is a compound according to formula (Ia), wherein the concentration refers to the ADM concentration comprised in the PEG-ADM;
water;
5.4 mg/mL of citric acid anhydrous,
2.2 mg/mL of sodium hydroxide,
8.1 mg/mL hydrochloric acid 10% (m/V); and
6.54 mg/mL of sodium chloride,

wherein the concentrations of the components are based on the total volume of the liquid pharmaceutical formulation; wherein the aqueous formulation has a pH of 3.5 to 4.5, preferably a pH 3 to 4, more preferably a pH of 4.

In one embodiment the pharmaceutical formulation according to the invention comprises
0.04 mg/mL to 10 mg/mL PEG-ADM, wherein the PEG-ADM is a compound according to formula (I) or (Ia),
0.5 mg/mL to 25 mg/mL of the pH regulator, and
0.1 mg/mL to 30 mg/mL of the osmolarity regulator.

In one embodiment the pharmaceutical formulation according to the invention comprises
0.04 mg/mL to 7.7 mg/mL PEG-ADM, wherein the PEG-ADM is a compound according to formula (I) or (Ia),
0.8 mg/mL to 15 mg/mL of the pH regulator, and
0.5 mg/mL to 15 mg/mL of the osmolarity regulator.

In one embodiment the pharmaceutical formulation according to the invention comprises
0.04 mg/mL to 7.7 mg/mL PEG-ADM, wherein the PEG-ADM is a compound according to formula (I) or (Ia),
0.8 mg/mL to 15 mg/mL of the pH regulator, and
2 mg/mL to 10 mg/mL of the osmolarity regulator.

In one embodiment the pharmaceutical formulation according to the invention comprises
0.2 mg/mL to 10 mg/mL PEG-ADM, wherein the PEG-ADM is a compound according to formula (I) or (Ia),
0.5 mg/mL to 25 mg/mL of the pH regulator, and
0.1 mg/mL to 30 mg/mL of the osmolarity regulator.

In one embodiment the pharmaceutical formulation according to the invention comprises
0.2 mg/mL to 7.7 mg/mL PEG-ADM, wherein the PEG-ADM is a compound according to formula (I) or (Ia),
0.8 mg/mL to 15 mg/mL of the pH regulator, and
0.5 mg/mL to 15 mg/mL of the osmolarity regulator.

In one embodiment the pharmaceutical formulation according to the invention comprises
0.2 mg/mL to 7.7 mg/mL PEG-ADM, wherein the PEG-ADM is a compound according to formula (I) or (Ia),
0.8 mg/mL to 15 mg/mL of the pH regulator, and
2 mg/mL to 10 mg/mL of the osmolarity regulator.

In one embodiment the pharmaceutical formulation according to the invention comprises
3.696 mg/mL PEG-ADM, wherein the PEG-ADM is a compound according to formula (Ia),
0.5 mg/mL to 25 mg/mL of the pH regulator, and
0.1 mg/mL to 30 mg/mL of the osmolarity regulator.

In one embodiment the pharmaceutical formulation according to the invention comprises
3.696 mg/mL PEG-ADM, wherein the PEG-ADM is a compound according to formula (Ia),
0.8 mg/mL to 15 mg/mL of the pH regulator, and
0.5 mg/mL to 15 mg/mL of the osmolarity regulator.

In one embodiment the pharmaceutical formulation according to the invention comprises
3.696 mg/mL PEG-ADM, wherein the PEG-ADM is a compound according to formula (Ia),
0.8 mg/mL to 15 mg/mL of the pH regulator, and
2 mg/mL to 10 mg/mL of the osmolarity regulator.

The embodiments disclosed in this section “Further embodiments” can also have the pH, the osmolar concentration and/or the viscosity as disclosed in sections “pH of the pharmaceutical formulation”, “viscosity of the pharmaceutical formulation” or “osmolar concentration of the pharmaceutical formulation”, respectively.

Excipients

The pharmaceutical formulation according to the invention or any embodiment disclosed herein can further comprise at least one excipient. In the context of the present invention, excipients are substances which, in the pharmaceutical formulation serve the purpose, for example, of microbiologically, chemically and physically stabilizing the preparation or improving the taste or optical appearance. The term “excipients” also comprises with an inert nontoxic pharmaceutically suitable excipient. Examples of excipients in the context of the present invention are antioxidants, stabilizers, preservatives, substances for adjusting tonicity, aromas, fragrances or dyes.

Combined Pharmaceutical Dosage Form

In one embodiment according to the invention, the combination is a combined pharmaceutical dose form. The “combined pharmaceutical dose form” is used to combine two or more pharmaceutical dose forms into a single term, in order to describe a medicinal product that consists of two or more manufactured items that are intended to be combined to produce a single pharmaceutical product for administration to the patient. A combined pharmaceutical dose form is not used to combine pharmaceutical dose forms that are packaged together but administered separately rather than being combined to produce a single pharmaceutical product (see instead combination packs). “Pharmaceutical dose form” and “dosage form” are synonyms. “Pharmaceutical dose form” or “dosage form” is the physical manifestation of a product that contains or comprises the active ingredient and/or inactive ingredients (e.g. carrier, excipients) that are intended to be delivered to the patient. “Dosage form” is the term used in the European Pharmacopoeia. “Dosage form” was previously used in Standard Terms, but the term “pharmaceutical dose form” is now used in order to harmonize with the vocabulary that is used across the Identification of Medicinal Products project (cf. https://www.edqm.eu/sites/default/files/standard_terms_introduction_and_guidance_for_use.pdf). Common dosage forms include pill, tablet, capsule, syrup, aerosol, liquid injection, powder, or solid crystal, and so on. Further pharmaceutical formulations or dosage forms are disclosed below. The route of administration for drug delivery is dependent on the dosage form of the active ingredient.

Combination Pack

One aspect of the present invention is a combination pack. In a “combination pack” the components are included in separate dosage forms marketed in the same package. A combination is different from a combined pharmaceutical dose form. In one embodiment, the combination pack comprises any one of the embodiments of the pharmaceutical formulation disclosed herein and a nebulizer. In one embodiment the nebulizer is a mesh nebulizer or vibrating mesh nebulizer. In one embodiment the nebulizer is an Aerogen Solo nebulizer optionally combined with a Aerogen Pro-X or Aerogen USB controller.

### Method for Preparing the Pharmaceutical Formulation

One subject of the invention is the preparation of the pharmaceutical formulation according to the invention.

The method comprises at least the following steps
step 1. Providing components a, b, c and d; and
step 2. Mixing the components provided in step 1;

whereby the following pharmaceutical formulation is obtained:
a liquid pharmaceutical formulation formulation comprising:
a. 0.04 mg/mL to 145 mg/mL of PEG-ADM, wherein the PEG-ADM is a compound according to the general formula (I),

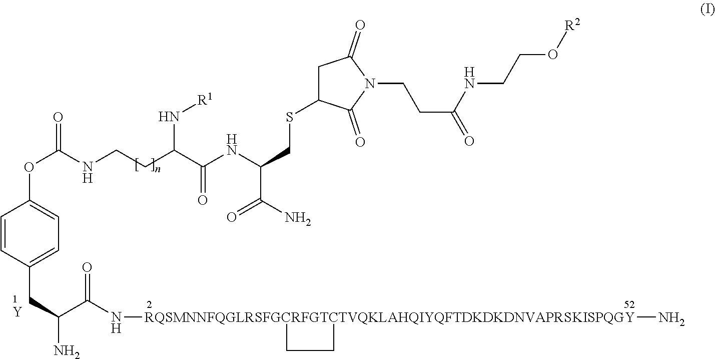

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
or a hydrate thereof, solvate thereof, salt thereof, pharmaceutically acceptable salt thereof, or the solvates of salts thereof;
b. a solvent;
c. a pH regulator; and
d. an osmolarity regulator;
wherein the pharmaceutical formulation has a pH of 3 to 5; and wherein the osmolar concentration is between 150 to 450 mosmol/L, and wherein the concentrations of the components are based on the total volume of the liquid pharmaceutical formulation.

Steps 1 and/or 2 can be conducted separately and/or simultaneously and/or subsequently.

In one embodiment of the method, the PEG-ADM (or component a) is a compound according to any one of the embodiments disclosed under section “PEG-ADM (component a)” above. In one embodiment of the method, the PEG-ADM is a compound according to formula (Ia). In one embodiment of the method, the component b is a compound according to any one of the embodiments disclosed under section “solvent (component b)” above. In one embodiment of the method, the component c is a compound according to any one of the embodiments disclosed under section “pH regulator (component c)” above. In one embodiment of the method, the component d is a compound according to any one of the embodiments disclosed under section “osmolarity regulator (component d)” above. In one embodiment of the method, the pharmaceutical formulation obtained is selected from the embodiments disclosed in the section “Further embodiments of the pharmaceutical formulation”.

In one embodiment of the method, the method further comprises step 3
step 3. adjusting the pH of the pharmaceutical formulation to a pH of 3 to 5,

wherein step 3 can be carried before, during and/or after step 1, 2 and/or step 4.

In one embodiment the pH can be adjusted to any pH disclosed under section “pH of the pharmaceutical formulation”. Steps 1 and/or 2 and/or 3 can be conducted separately and/or simultaneously and/or subsequently. Steps 1 and/or 2 and/or 3 and/or 4 can be conducted separately and/or simultaneously and/or subsequently.

In one embodiment of the method, the method further comprises step 4
step 4. Adjusting the osmolarity of the pharmaceutical formulation to an osmotic concentration of 150 to 450 mosmol/l;

wherein step 4 can be carried before, during and/or after step 1, 2 and/or step 3.

Steps 1 and/or 2 and/or 3 and/or 4 can be conducted separately and/or simultaneously and/or subsequently.

In one embodiment of the method, the method comprises steps 1 to 4 and the pharmaceutical formulation is prepared as follows
providing an aqueous formulation of PEG-ADM, which comprises citric acid and optionally at least one pH regulator to adjust the pH to 3.5 and 4.5,
followed by concentration of the aqueous formulation of PEG-ADM and
subsequently reconstitution/dilution of the concentrated product by adding a solution of citric acid and/or sodium citrate, optionally at least one pH regulator and an osmolarity regulator and water, and

wherein the pharmaceutical formulation has an osmotic concentration of 150 to 450 mosmol/l mosmol/l;

and wherein the pH of the resulting aqueous formulation is between 3.5 and 4.5.

In one embodiment of the method, the method comprises steps 1 to 4 and the pharmaceutical formulation is prepared as follows
providing an aqueous formulation of PEG-ADM, which comprises citric acid and optionally at least one pH regulator to adjust the pH to 3.5 and 4.5,
providing citric acid and/or sodium citrate, optionally at least one pH regulator and an osmolarity regulator and
mixing the solutions provided, and

wherein the pharmaceutical formulation has an osmotic concentration of 150 to 450 mosmol/l mosmol/l; and wherein the pH of the resulting aqueous formulation is between 3.5 and 4.5.

The method according to the invention or the embodiments thereof can further comprise step 5:

Step 5 at least partially freezing the pharmaceutical formulation obtained after any one of steps 1, 2, 3 and/or 4.

Steps 1 and/or 2 and/or 3 and/or 4 and/or 5 can be conducted separately and/or simultaneously and/or subsequently.

Indications

In one embodiment the pharmaceutical formulation according to the invention and the compounds according to formula (I) or (Ia) are suitable for treatment and/or prevention of pulmonary disorders, such as pulmonary hypertension; secondary pulmonary hypertension; pulmonary hypertension following pulmonary embolism with and without acute cor pulmonale; primary pulmonary hypertension; chronic obstructive pulmonary disease; asthma; acute pulmonary edema; chronic pulmonary edema; allergic alveolitis; pneumonitis due to inhaled organic dust; pneumonitis due to inhaled particles of fungal, actinomycetic or other origin; acute chemical bronchitis; acute chemical pulmonary edema and/or chronic chemical pulmonary edema (e.g. after inhalation of phosgene, nitrogen oxide); neurogenic pulmonary edema; acute pulmonary manifestations due to radiation; chronic pulmonary manifestations due to radiation; acute and/or chronic interstitial lung disorders (such as but not restricted to drug-induced interstitial lung disorders, e.g. secondary to Bleomycin treatment); acute lung injury (ALI); acute lung injury (ALI) in adult or child including newborn; acute respiratory distress syndrome (ARDS); acute respiratory distress syndrome (ARDS) in adult or child including newborn; ALI/ARDS secondary to pneumonia and sepsis, aspiration pneumonia and ALI/ARDS secondary to aspiration (such as but not restricted to aspiration pneumonia due to regurgitated gastric content); ALI/ARDS secondary to smoke gas inhalation; transfusion-related acute lung injury (TRALI), ALI/ARDS or acute pulmonary insufficiency following surgery; trauma or burns, ventilator induced lung injury (VILI); lung injury following meconium aspiration; pulmonary fibrosis; and mountain sickness.

In one embodiment the pharmaceutical formulation according to the invention and the compounds according to formula (I) or (Ia) are suitable for treatment and/or prevention of ALI/ARDS secondary to pneumonia caused by bacterial infection of the lungs, such as, but not restricted to, bacterial pneumonia caused by Pneumococci, Haemophilus influenzae, Mycoplasma pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria.

In one embodiment the pharmaceutical formulation according to the invention and the compounds according to formula (I) or (Ia) are suitable for treatment and/or prevention of ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV).

In one embodiment the pharmaceutical formulation according to the invention and the compounds according to formula (I) or (Ia) are also suitable for treatment and/or prevention of ALI/ARDS secondary to pneumonia caused by fungal infections such as, but not restricted to, fungal pneumonia caused by Pneumocystis Jirovecii.

In one embodiment the pharmaceutical formulation according to the invention and the compounds according to formula (I) or (Ia) are suitable for treatment and/or prevention of ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP).

In one embodiment the pharmaceutical formulation according to the invention and the compounds according to formula (I) or (Ia) are suitable for treatment and/or prevention of ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue).

In one embodiment the pharmaceutical formulation according to the invention and the compounds according to formula (I) or (Ia) are suitable for treatment and/or prevention of ALI/ARDS secondary to pneumonia occurring in consequence of bacterial and/or virus infection.

In one embodiment the pharmaceutical formulation according to the invention and the compounds according to formula (I) or (Ia) are suitable for treatment and/or prevention of ALI/ARDS secondary to pneumonia occurring in consequence of a bacterial superinfection of a primary lung affection by viruses.

In one embodiment the pharmaceutical formulation according to the invention and the compounds according to formula (I) or (Ia) are suited for the prevention and/or treatment of lung dysfunction after lung transplantations.

On the basis of their pharmacological properties, the pharmaceutical formulation according to the invention and the compounds according to formula (I) or (Ia) according to the invention can be employed to prevent and/or ameliorate development of sepsis secondary to bacterial pneumonia (so called pneumogenic sepsis).

A further embodiment is the compound according to formula (I) or the compound according to formula (Ia) for use in the treatment and/or prevention of the disorders and/or diseases listed in this section “Indications”. The pharmaceutical formulation according to the invention and the compounds according to formula (I) or (Ia) are in particular suitable for treatment and/or prevention of ALI/ARDS in immunocompromised patients suffering from pneumonia, such as in the context of acquired immunodeficiency syndrome (AIDS), chemotherapy and bone marrow transplantation.

### Clauses

The following clauses also form part of the disclosure and refer to further embodiments of the invention:
1. Liquid pharmaceutical formulation comprising:
   a. 0.04 mg/mL to 145 mg/mL of PEG-ADM, wherein the PEG-ADM is a compound according to the general formula (I),

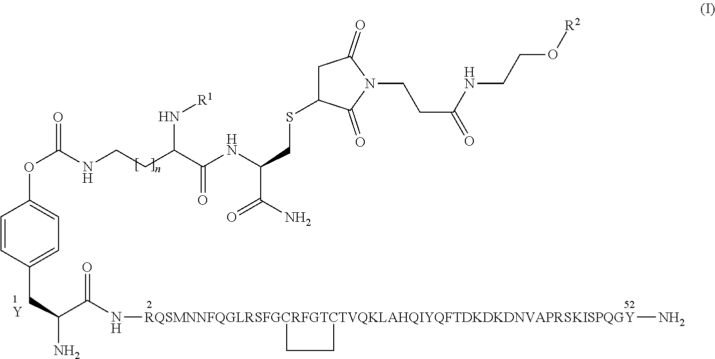

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group, or a hydrate thereof, solvate thereof, salt thereof, pharmaceutically acceptable salt thereof, or the solvates of salts thereof;
b. a solvent;
c. a pH regulator; and
d. an osmolarity regulator;
wherein the pharmaceutical formulation has a pH of 3 to 5; and wherein the osmolar concentration is between 150 to 450 mosmol/L, and
wherein the concentrations of the components are based on the total volume of the liquid pharmaceutical formulation.
2. The pharmaceutical formulation according to clause 1, wherein the pharmaceutical formulation is a solution or a dispersion.
3. The pharmaceutical formulation according to any one of clauses 1 or 2, wherein the pharmaceutical formulation is a frozen solution or frozen dispersion.
4. The pharmaceutical formulation according to any one of the preceding clauses, wherein the pharmaceutical formulation is an aqueous solution.
5. The pharmaceutical formulation according to any one of the preceding clauses, wherein the PEG-ADM is selected from compounds of the general formula (I) and R² represents linear or branched PEG 20 kDa endcapped with a methoxy-group, wherein the PEG-ADM is a compound according to the general formula (I) as defined in any one of the preceding clauses, or a hydrate thereof, solvate thereof, salt thereof, pharmaceutically acceptable salt thereof, or the solvates of salts thereof.
6. The pharmaceutical formulation according to any one of the preceding clauses, wherein the PEG-ADM is selected from compounds of the general formula (I) and R² represents linear or branched PEG 40 kDa endcapped with a methoxy-group, wherein the PEG-ADM is a compound according to the general formula (I) as defined in any one of the preceding clauses, or a hydrate thereof, solvate thereof, salt thereof, pharmaceutically acceptable salt thereof, or the solvates of salts thereof.
7. The pharmaceutical formulation according to any one of the preceding clauses, wherein the PEG-ADM is selected from compounds of the general formula (I) and R² represents linear or branched PEG 80 kDa endcapped with a methoxy-group, wherein the PEG-ADM is a compound according to the general formula (I) as defined in any one of the preceding clauses, or a hydrate thereof, solvate thereof, salt thereof, pharmaceutically acceptable salt thereof, or the solvates of salts thereof.
8. The pharmaceutical formulation according to any one of the preceding clauses, wherein the PEG-ADM is selected from compounds of the general formula (I),

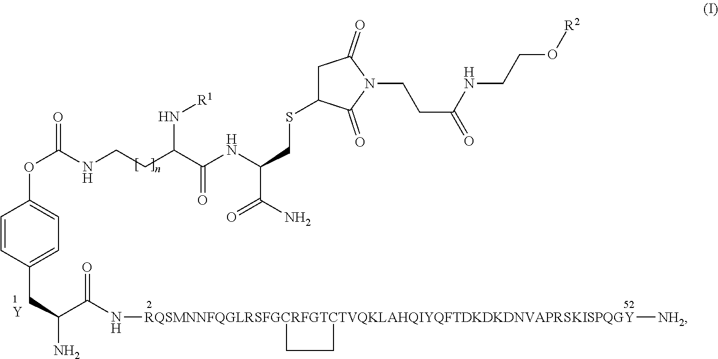

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group, or a hydrate thereof, solvate thereof, salt thereof, pharmaceutically acceptable salt thereof, or the solvates of salts thereof.
9. The pharmaceutical formulations according to any one of the preceding clauses, wherein the PEG-ADM is selected from compounds of the formula (I) in which
   n represents the number 1 or 2,
   R¹ represents hydrogen or methyl,
   R² represents linear PEG 40 kDa endcapped with a methoxy-group.
10. The pharmaceutical formulations according to any one of the preceding clauses, wherein the PEG-ADM is selected from compounds of the formula (I) in which
   n represents the number 1 or 2,
   R¹ represents hydrogen,
   R² represents linear PEG 40 kDa endcapped with a methoxy-group.
11. The pharmaceutical formulation according to any one of the preceding clauses, wherein the PEG-ADM is the compound according to formula (Ia)

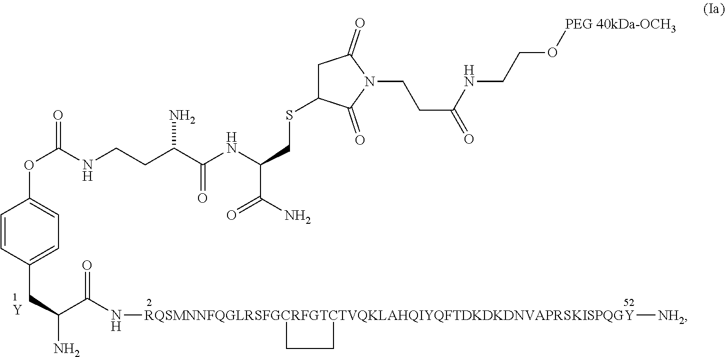

12. The pharmaceutical formulation according to any one of the preceding clauses, wherein the pharmaceutical formulation comprises 0.385 mg/mL to 77 mg/mL PEG-ADM.
13. The pharmaceutical formulation according to any one of the preceding clauses, wherein the pharmaceutical formulation comprises 2.31 mg/mL to 77 mg/mL PEG-ADM.
14. The pharmaceutical formulation according to any one of the preceding clauses, wherein the pharmaceutical formulation comprises 3.85 mg/mL to 77 mg/mL PEG-ADM.
15. The pharmaceutical formulation according to any one of the preceding clauses, wherein, wherein the pharmaceutical formulation comprises 7.7 mg/mL to 77 mg/mL PEG-ADM.
16. The pharmaceutical formulation according to any one of the preceding clauses, wherein the pharmaceutical formulation comprises 0.385 mg/mL to 38.5 mg/mL PEG-ADM.
17. The pharmaceutical formulation according to any one of the preceding clauses, wherein the pharmaceutical formulation comprises 0.77 mg/mL to 38.5 mg/mL PEGADM.
18. The pharmaceutical formulation according to any one of the preceding clauses, wherein the pharmaceutical formulation comprises 0.77 mg/mL to 23.1 mg/mL PEGADM.
19. The pharmaceutical formulation according to any one of the preceding clauses, wherein the pharmaceutical formulation comprises 0.77 mg/mL to 7.7 mg/mL PEG-ADM.
20. The pharmaceutical formulation according to any one of the preceding clauses, wherein the pharmaceutical formulation comprises 2.31 mg/mL to 7.7 mg/mL PEG-ADM.
21. The pharmaceutical formulation according to any one of the preceding clauses, wherein the pharmaceutical formulation comprises 2.31 mg/mL to 3.85 mg/mL PEG-ADM.
22. The pharmaceutical formulation according to any one of the preceding clauses, wherein the pharmaceutical formulation comprises 3.08 mg/mL to 23.1 mg/mL PEG-ADM.
23. The pharmaceutical formulation according to any one of the preceding clauses, wherein the pharmaceutical formulation comprises 3.08 mg/mL to 7.7 mg/mL PEG-ADM.
24. The pharmaceutical formulation according to any one of the preceding clauses, wherein the pharmaceutical formulation comprises 0.77 mg/mL PEG-ADM.
25. The pharmaceutical formulation according to any one of the preceding clauses, wherein the pharmaceutical formulation comprises 6.16 mg/mL PEG-ADM.
26. The pharmaceutical formulation according to any one of the preceding clauses, wherein the pharmaceutical formulation comprises 4.6 g/mL PEG-ADM.
27. The pharmaceutical formulation according to any one of the preceding clauses, wherein the pharmaceutical formulation comprises 3.85 mg/mL PEG-ADM.

28. The pharmaceutical formulation according to any one of the preceding clauses, wherein the pharmaceutical formulation comprises 3.7 mg/mL PEG-ADM.
29. The pharmaceutical formulation according to any one of the preceding clauses, wherein the pharmaceutical formulation comprises 2.31 mg/mL PEG-ADM.
30. The pharmaceutical formulation according to any one of the preceding clauses, wherein the pharmaceutical formulation comprises approximately 0.044 mg/mL to 44 mg/mL PEG-ADM.
31. The pharmaceutical formulation according to any one of the preceding clauses, wherein the pharmaceutical formulation comprises approximately 0.22 mg/mL to 22 mg/mL PEG-ADM.
32. The pharmaceutical formulation according to any one of the preceding clauses, wherein the pharmaceutical formulation comprises approximately 0.44 mg/mL to 13.2 mg/mL PEG-ADM.
33. The pharmaceutical formulation according to any one of the preceding clauses, wherein the pharmaceutical formulation comprises approximately 0.44 mg/mL to 4.4 mg/mL PEG-ADM.
34. The pharmaceutical formulation according to any one of the preceding clauses, wherein the pharmaceutical formulation comprises approximately 1.3 mg/mL to 2.2 mg/mL PEG-ADM.
35. The pharmaceutical formulation according to any one of the preceding clauses, wherein the pharmaceutical formulation comprises approximately 0.14 mg/mL to 144 mg/mL PEG-ADM.
36. The pharmaceutical formulation according to any one of the preceding clauses, wherein the pharmaceutical formulation comprises approximately 0.7 mg/mL to 71.7 mg/mL PEG-ADM.
37. The pharmaceutical formulation according to any one of the preceding clauses, wherein the pharmaceutical formulation comprises approximately 1.4 mg/mL to 43 mg/mL PEG-ADM.
38. The pharmaceutical formulation according to any one of the preceding clauses, wherein the pharmaceutical formulation comprises approximately 1.4 mg/mL to 14.3 mg/mL PEG-ADM.
39. The pharmaceutical formulation according to any one of the preceding clauses, wherein the pharmaceutical formulation comprises approximately 4.3 mg/mL to 7.2 mg/mL PEG-ADM.
40. The pharmaceutical formulation according to any one of the preceding clauses, wherein the solvent comprises water.
41. The pharmaceutical formulation according to any one of the preceding clauses, wherein the solvent is water.
42. The pharmaceutical formulation according to any one of the preceding clauses, wherein the solvent is substantially water.
43. The pharmaceutical formulation according to any one of the preceding clauses, wherein the pharmaceutical formulation comprises 0.1 mg/mL to 250 mg/mL of the pH regulator.
44. The pharmaceutical formulation according to any one of the preceding clauses, wherein the pharmaceutical formulation comprises 0.3 mg/mL to 250 mg/mL of the pH regulator.
45. The pharmaceutical formulation according to any one of the preceding clauses, wherein the pharmaceutical formulation comprises 0.5 mg/mL to 100 mg/mL of the pH regulator.
46. The pharmaceutical formulation according to any one of the preceding clauses, wherein the pharmaceutical formulation comprises 0.9 mg/mL to 90 mg/mL of the pH regulator.
47. The pharmaceutical formulation according to any one of the preceding clauses, wherein the pharmaceutical formulation comprises 2.5 mg/mL to 46 mg/mL of the pH regulator.
48. The pharmaceutical formulation according to any one of the preceding clauses, wherein the pharmaceutical formulation comprises 7.8 mg/mL to 29 mg/mL of the pH regulator.
49. The pharmaceutical formulation according to any one of the preceding clauses, wherein the pharmaceutical formulation comprises 12.5 mg/mL to 19 mg/mL of the pH regulator.
50. The pharmaceutical formulation according to any one of the preceding clauses, wherein the pharmaceutical formulation comprises 0.01 mg/mL to 100 mg/mL of the pH regulator.
51. The pharmaceutical formulation according to any one of the preceding clauses, wherein the pharmaceutical formulation comprises 0.1 mg/mL to 50 mg/mL of the pH regulator.
52. The pharmaceutical formulation according to any one of the preceding clauses, wherein the pharmaceutical formulation comprises 0.5 mg/mL to 25 mg/mL of the pH regulator.
53. The pharmaceutical formulation according to any one of the preceding clauses, wherein the pharmaceutical formulation comprises 0.8 mg/mL to 15 mg/mL of the pH regulator.
54. The pharmaceutical formulation according to any one of the preceding clauses, wherein the pharmaceutical formulation comprises 1.5 mg/mL to 9 mg/mL of the pH regulator.
55. The pharmaceutical formulation according to any one of the preceding clauses, wherein the pH regulator comprises citric acid, a salt of citric acid, a pharmaceutical acceptable salt of citric acid, a derivative of citric acid, and/or mixtures thereof.
56. The pharmaceutical formulation according to any one of the preceding clauses, wherein the pH regulator comprises hydrochloric acid, citric acid, a salt of citric acid, pharmaceutical acceptable salt of citric acid, derivative of citric acid, and/or mixtures thereof.
57. The pharmaceutical formulation according to any one of the preceding clauses, wherein the pH regulator comprises hydrochloric acid.
58. The pharmaceutical formulation according to any one of the preceding clauses, wherein the pH regulator comprises a mixture comprising hydrochloric acid and sodium hydroxide.
59. The pharmaceutical formulation according to any one of the preceding clauses, wherein the pH regulator comprises a mixture comprising hydrochloric acid, sodium hydroxide and citric acid.
60. The pharmaceutical formulation according to any one of the preceding clauses, wherein the pH regulator comprises a mixture comprising sodium hydroxide and citric acid.
61. The pharmaceutical formulation according to any one of the preceding clauses, wherein the pH regulator comprises a mixture comprising sodium citrate and hydrochloric acid.
62. The pharmaceutical formulation according to any one of the preceding clauses, wherein the pH regulator consists of hydrochloric acid.
63. The pharmaceutical formulation according to any one of the preceding clauses, wherein the pH regulator consists of a mixture comprising hydrochloric acid and sodium hydroxide.
64. The pharmaceutical formulation according to any one of the preceding clauses, wherein the pH regulator consists of a mixture comprising hydrochloric acid, sodium hydroxide and citric acid.
65. The pharmaceutical formulation according to any one of the preceding clauses, wherein the pH regulator consists of a mixture comprising sodium hydroxide and citric acid.
66. The pharmaceutical formulation according to any one of the preceding clauses, wherein the pH regulator consists of a mixture comprising sodium citrate and hydrochloric acid.
67. The pharmaceutical formulation according to any one of the preceding clauses, wherein the citric acid is a salt of citric acid, pharmaceutical acceptable salt of citric acid, derivative of citric acid, and/or mixtures thereof.
68. The pharmaceutical formulation according to any one of the preceding clauses, wherein the salt of citric acid, pharmaceutical acceptable salt of citric acid, derivative of citric acid, and/or mixtures thereof is selected from the group consisting of citric acid anhydrous, sodium citrate and citric acid monohydrate.
69. The pharmaceutical formulation according to any one of the preceding clauses, wherein the pH regulator comprises or consists of hydrochloric acid, preferably hydrochloric acid.
70. The pharmaceutical formulation according to any one of the preceding clauses, wherein the pH regulator comprises or consists of 0.1 mg/mL to 100 mg/mL citric acid.
71. The pharmaceutical formulation according to any one of the preceding clauses, wherein the pH regulator comprises or consists of 0.3 mg/mL to 30 mg/mL citric acid.
72. The pharmaceutical formulation according to any one of the preceding clauses, wherein the pH regulator comprises or consists of 1 mg/mL to 15 mg/mL citric acid.
73. The pharmaceutical formulation according to any one of the preceding clauses, wherein the pH regulator comprises or consists of 2 mg/mL to 10 mg/mL citric acid.
74. The pharmaceutical formulation according to any one of the preceding clauses, wherein the pH regulator comprises or consists of 4 mg/mL to 7 mg/mL citric acid.
75. The pharmaceutical formulation according to any one of the preceding clauses, wherein the pH regulator comprises or consists of wherein the pH regulator comprises or consists of 0.01 mg/mL to 50 mg/mL sodium hydroxide.
76. The pharmaceutical formulation according to any one of the preceding clauses, wherein the pH regulator comprises or consists of 0.1 mg/mL to 10 mg/mL sodium hydroxide.
77. The pharmaceutical formulation according to any one of the preceding clauses, wherein the pH regulator comprises or consists of 0.5 mg/mL to 6 mg/mL sodium hydroxide.
78. The pharmaceutical formulation according to any one of the preceding clauses, wherein the pH regulator comprises or consists of 0.8 mg/mL to 4 mg/mL sodium hydroxide.
79. The pharmaceutical formulation according to any one of the preceding clauses, wherein the pH regulator comprises or consists of 0.1 mg/mL to 100 mg/mL hydrochloric acid.
80. The pharmaceutical formulation according to any one of the preceding clauses, wherein the pH regulator comprises or consists of 0.5 mg/mL to 50 mg/mL hydrochloric acid.
81. The pharmaceutical formulation according to any one of the preceding clauses, wherein the pH regulator comprises or consists of 1 mg/mL to 25 mg/mL hydrochloric acid.
82. The pharmaceutical formulation according to any one of the preceding clauses, wherein the pH regulator comprises or consists of 5 mg/mL to 15 mg/mL of hydrochloric acid 10% (m/V).
83. The pharmaceutical formulation according to any one of the preceding clauses, wherein the pharmaceutical formulation comprises two or more pH regulators.
84. The pharmaceutical formulation according to any one of the preceding clauses, wherein the pharmaceutical formulation comprises three or more pH regulators.
85. The pharmaceutical formulation according to any one of the preceding clauses, wherein the osmolarity regulator is selected from the group consisting of sodium chloride, citric acid, a salt, pharmaceutical acceptable salt, derivative of citric acid and/or mixtures thereof.
86. The pharmaceutical formulation according to any one of the preceding clauses, wherein the citric acid is a salt, pharmaceutical acceptable salt, derivative of citric acid is selected from the group consisting of citric acid anhydrous, sodium citrate and citric acid monohydrate.
87. The pharmaceutical formulation according to any one of the preceding clauses, wherein the osmolarity regulator is sodium chloride.
88. The pharmaceutical formulation according to any one of the preceding clauses, wherein the pharmaceutical formulation comprises 0.01 mg/mL to 100 mg/mL of the osmolarity regulator.
89. The pharmaceutical formulation according to any one of the preceding clauses, wherein the pharmaceutical formulation comprises 0.1 mg/mL to 30 mg/mL of the osmolarity regulator.
90. The pharmaceutical formulation according to any one of the preceding clauses, wherein the pharmaceutical formulation comprises 0.5 mg/mL to 15 mg/mL of the osmolarity regulator.
91. The pharmaceutical formulation according to any one of the preceding clauses, wherein the pharmaceutical formulation comprises 2 mg/mL to 10 mg/mL of the osmolarity regulator or 5 mg/mL to 7 mg/mL of the osmolarity regulator.
92. The pharmaceutical formulation according to any one of the preceding clauses, wherein the pharmaceutical formulation has an osmotic concentration between 150 mosmol/l to 450 mosmol/L or 200 to 400 mosmol/l.
93. The pharmaceutical formulation according to any one of the preceding clauses, wherein the pharmaceutical formulation has an osmotic concentration between 270 to 330 mosmol/l.
94. The pharmaceutical formulation according to any one of the preceding clauses, wherein the pharmaceutical formulation has an osmotic concentration between 250 to 310 mosmol/l.
95. The pharmaceutical formulation according to any one of the preceding clauses, wherein the pharmaceutical formulation has an osmotic concentration of 300 mosmol/l.
96. The pharmaceutical formulation according to any one of the preceding clauses, wherein the formulation has a pH of 3.5 to 4.5.
97. The pharmaceutical formulation according to any one of the preceding clauses, wherein the formulation has a pH of 3 to 4.
98. The pharmaceutical formulation according to any one of the preceding clauses, wherein the formulation has a pH of 3 to 3.5.
99. The pharmaceutical formulation according to any one of the preceding clauses, wherein the formulation has a pH of 3.5 to 4.
100. The pharmaceutical formulation according to any one of the preceding clauses, wherein the formulation has a pH of 3.5.
101. The pharmaceutical formulation according to any one of the preceding clauses, wherein the formulation has a pH of 4.
102. The pharmaceutical formulation according to any one of the preceding clauses, wherein the formulation has a viscosity of 0.9 to 2.2 mPa*s, 1 to 2 mPa*s, 1.05 to 2 mPa*s, 1.1 to 2 mPa*s or 1.05 to 1.9 mPa*s.
103. The pharmaceutical formulation according to any one of the preceding clauses, wherein the pharmaceutical formulation comprises as pH regulator
   0.1 mg/mL to 100 mg/mL citric acid;
   0.01 mg/mL to 50 mg/mL sodium hydroxide;
   0.1 mg/mL to 100 mg/mL hydrochloric acid.
104. The pharmaceutical formulation according to any one of the preceding clauses, wherein the pharmaceutical formulation comprises as pH regulator
   0.3 mg/mL to 30 mg/mL citric acid;
   0.1 mg/mL to 10 mg/mL sodium hydroxide;
   0.5 mg/mL to 50 mg/mL hydrochloric acid.
105. The pharmaceutical formulation according to any one of the preceding clauses, wherein the pharmaceutical formulation comprises as pH regulator
   1 mg/mL to 15 mg/mL citric acid;
   0.5 mg/mL to 6 mg/mL sodium hydroxide;
   1 mg/mL to 25 mg/mL hydrochloric acid.
106. The pharmaceutical formulation according to any one of the preceding clauses, wherein the pharmaceutical formulation comprises as pH regulator
   2 mg/mL to 10 mg/mL citric acid;
   0.8 mg/mL to 4 mg/mL sodium hydroxide;
   5 mg/mL to 15 mg/mL hydrochloric acid.
107. The pharmaceutical formulation according to any one of the preceding clauses, wherein the pharmaceutical formulation comprises
   0.077 mg/mL to 77 mg/mL PEG-ADM,
   0.1 mg/mL to 100 mg/mL citric acid;
   0.01 mg/mL to 50 mg/mL sodium hydroxide;
   0.1 mg/mL to 100 mg/mL hydrochloric acid.
108. The pharmaceutical formulation according to any one of the preceding clauses, wherein the pharmaceutical formulation comprises
   0.385 mg/mL to 3.85 mg/mL PEG-ADM,
   0.3 mg/mL to 30 mg/mL citric acid;
   0.1 mg/mL to 10 mg/mL sodium hydroxide;
   0.5 mg/mL to 50 mg/mL hydrochloric acid.
109. The pharmaceutical formulation according to any one of the preceding clauses, wherein the pharmaceutical formulation comprises
   0.77 mg/mL to 23.1 mg/mL PEG-ADM,
   1 mg/mL to 15 mg/mL citric acid;
   0.5 mg/mL to 6 mg/mL sodium hydroxide;
   1 mg/mL to 25 mg/mL hydrochloric acid.
110. The pharmaceutical formulation according to any one of the preceding clauses, wherein the pharmaceutical formulation comprises
   0.77 mg/mL to 7.7 mg/mL PEG-ADM,
   2 mg/mL to 10 mg/mL citric acid;
   0.8 mg/mL to 4 mg/mL sodium hydroxide; and
   5 mg/mL to 15 mg/mL hydrochloric acid.
111. The pharmaceutical formulation according to any one of the preceding clauses, wherein the pharmaceutical formulation comprises
   2.31 mg/mL to 3.85 mg/mL PEG-ADM,
   4 mg/mL to 7 mg/mL citric acid;
   1.5 mg/mL to 3 mg/mL sodium hydroxide; and
   7 mg/mL to 9 mg/mL hydrochloric acid.
112. The pharmaceutical formulation according to any one of the preceding clauses, wherein the pharmaceutical formulation comprises
   0.077 mg/mL to 77 mg/mL PEG-ADM,
   0.1 mg/mL to 100 mg/mL citric acid;
   0.01 mg/mL to 50 mg/mL sodium hydroxide;
   0.1 mg/mL to 100 mg/mL hydrochloric acid;
   0.01 mg/mL to 100 mg/mL sodium chloride.
113. The pharmaceutical formulation according to any one of the preceding clauses, wherein the pharmaceutical formulation comprises
   0.385 mg/mL to 3.85 mg/mL PEG-ADM,
   0.3 mg/mL to 30 mg/mL citric acid;
   0.1 mg/mL to 10 mg/mL sodium hydroxide;
   0.5 mg/mL to 50 mg/mL hydrochloric acid;
   0.1 mg/mL to 30 mg/mL sodium chloride.
114. The pharmaceutical formulation according to any one of the preceding clauses, wherein the pharmaceutical formulation comprises
   0.77 mg/mL to 23.1 mg/mL PEG-ADM,
   1 mg/mL to 15 mg/mL citric acid;
   0.5 mg/mL to 6 mg/mL sodium hydroxide;
   1 mg/mL to 25 mg/mL hydrochloric acid;
   0.5 mg/mL to 15 mg/mL sodium chloride.
115. The pharmaceutical formulation according to any one of the preceding clauses, wherein the pharmaceutical formulation comprises
   0.77 mg/mL to 7.7 mg/mL PEG-ADM,
   2 mg/mL to 10 mg/mL citric acid;
   0.8 mg/mL to 4 mg/mL sodium hydroxide;
   5 mg/mL to 15 mg/mL hydrochloric acid;
   2 mg/mL to 10 mg/mL sodium chloride.
116. The pharmaceutical formulation according to any one of the preceding clauses, wherein the pharmaceutical formulation comprises
   2.31 mg/mL to 3.85 mg/mL PEG-ADM,
   4 mg/mL to 7 mg/mL citric acid;
   1.5 mg/mL to 3 mg/mL sodium hydroxide;
   7 mg/mL to 9 mg/mL hydrochloric acid;
   5 mg/mL to 7 mg/mL sodium chloride.
117. The pharmaceutical formulation according to any one of the preceding clauses, wherein the citric acid is a salt, pharmaceutical acceptable salt, derivative of citric acid is selected from the group consisting of citric acid anhydrous, sodium citrate and citric acid monohydrate.
118. The pharmaceutical formulation according to any one of the preceding clauses, wherein the hydrochloric acid is hydrochloric acid.
119. Pharmaceutical formulation according to any of clauses 1 to 118 for inhalation.
120. Medicament comprising the pharmaceutical formulation according to any one of clauses 1 to 119 or a medicament comprising the pharmaceutical formulation according to any one of clauses 1 to 119 in combination with an inert nontoxic pharmaceutically suitable excipient, optionally in combination with a further active ingredient.
121. Combined pharmaceutical dose form comprising components (1) and (2), wherein
   component (1) comprises a pharmaceutical formulation comprising PEG-ADM, a compound for adjusting the pH and an osmolarity regulator; wherein the pharmaceutical formulation comprising PEG-ADM, a compound for adjusting the pH and optionally a osmolarity regulator as defined according to any one of clauses 1 to 119; and component (2) comprises a solvent as defined in any one of clauses 1 to 119.
122. The combined pharmaceutical dose form according to clause 121, wherein the component (1) is solution, aqueous formulation, lyophilizate or frozen solution.
123. The combined pharmaceutical dose form according to clause 121 or 122, wherein the component (1) is a solution, dispersion, soluble powder, lyophilizate, tablet or granulate, which comprises at least one of the components a, c and/or component d, and component (2) comprises component b for solving or dispersing component (1).
124. Combination pack comprising component (1) and (2), wherein
   component (1) comprises the pharmaceutical formulation according to any one of clauses 1 to 119, the medicament according to clause 120, or the combined pharmaceutical dose form according to any one of clauses 121 to 123; and
   component (2) comprises a nebulizer, preferably a mesh nebulizer.
125. The pharmaceutical formulation according to any one of clauses 1 to 119, the medicament according to clause 120, the combined pharmaceutical dose form according to any one of clauses 121 to 123, or the combination pack according to clause 124 for use in the treatment and/or prevention of diseases.
126. The pharmaceutical formulation according to any one of clauses 1 to 119, the medicament according to clause 120, or the combined pharmaceutical dose form according to any one of clauses 121 to 123, the combination pack according to clause 124, the compound of formula (I) as defined in any one of clauses 1 to 119, the compound according to formula (Ia) as defined in any one of clauses 1 to 119 for use in the treatment and/or prevention of diseases and/or disorders, wherein the disease and/or disorder is selected from
   pulmonary disorders, such as pulmonary hypertension; secondary pulmonary hypertension; pulmonary hypertension following pulmonary embolism with and without acute cor pulmonale; primary pulmonary hypertension; chronic obstructive pulmonary disease; asthma; acute pulmonary edema; chronic pulmonary edema; allergic alveolitis; pneumonitis due to inhaled organic dust; pneumonitis due to inhaled particles of fungal, actinomycetic or other origin; acute chemical bronchitis; acute chemical pulmonary edema and/or chronic chemical pulmonary edema (e.g. after inhalation of phosgene, nitrogen oxide); neurogenic pulmonary edema; acute pulmonary manifestations due to radiation; chronic pulmonary manifestations due to radiation; acute and/or chronic interstitial lung disorders (such as but not restricted to drug-induced interstitial lung disorders, e.g. secondary to Bleomycin treatment); acute lung injury (ALI); acute lung injury (ALI) in adult or child including newborn; acute respiratory distress syndrome (ARDS); acute respiratory distress syndrome (ARDS) in adult or child including newborn; ALI/ARDS secondary to pneumonia and sepsis, aspiration pneumonia and ALI/ARDS secondary to aspiration (such as but not restricted to aspiration pneumonia due to regurgitated gastric content); ALI/ARDS secondary to smoke gas inhalation; transfusion-related acute lung injury (TRALI), ALI/ARDS or acute pulmonary insufficiency following surgery; trauma or burns, ventilator induced lung injury (VILI); lung injury following meconium aspiration; pulmonary fibrosis; and mountain sickness;
   ALI/ARDS secondary to pneumonia caused by bacterial infection of the lungs, such as, but not restricted to, bacterial pneumonia caused by Pneumococci, Haemophilus influenzae, Mycoplasma Pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria;
   ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV);
   ALI/ARDS secondary to pneumonia caused by fungal infections such as, but not restricted to, fungal pneumonia caused by Pneumocystis Jirovecii;
   ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP);
   ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue);
   ALI/ARDS secondary to pneumonia occurring in consequence of bacterial and/or virus infection;
   ALI/ARDS secondary to pneumonia occurring in consequence of a bacterial superinfection of a primary lung affection by viruses; and
   prevention and/or treatment of lung dysfunction after lung transplantations.
127. The compound of formula (I)

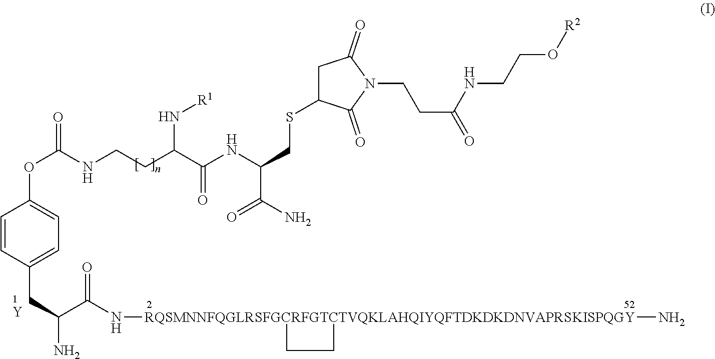

in which
n represents the number 0, 1, 2 or 3, R′ represents hydrogen, methyl, ethyl, n-propyl or isopropyl; R² represents linear or branched PEG 20 kDa to 80 kDa endcapped with a methoxy-group; or a hydrate thereof, solvate thereof, salt thereof, pharmaceutically acceptable salt thereof, or the solvates of salts thereof; as defined in any one of clauses 1 to 119 or the compound according to formula (Ia)

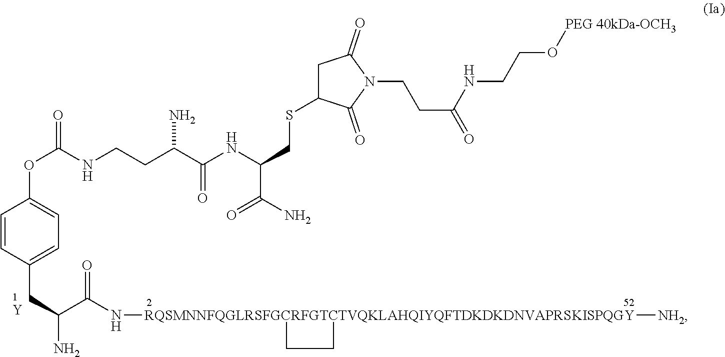

as defined in any one of clauses 1 to 119 for use in the treatment and/or prevention of ALI/ARDS secondary to pneumonia caused by bacterial infection of the lungs, such as, but not restricted to, bacterial pneumonia caused by Pneumococci, Haemophilus influenzae, Mycoplasma pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria; ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV); ALI/ARDS secondary to pneumonia caused by fungal infections such as, but not restricted to, fungal pneumonia caused by Pneumocystis Jirovecii; ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP); ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue); ALI/ARDS secondary to pneumonia occurring in consequence of bacterial and/or virus infection; and ALI/ARDS secondary to pneumonia occurring in consequence of a bacterial superinfection of a primary lung affection by viruses.
128. The use of the pharmaceutical formulation according to any one of clauses 1 to 119, the medicament according to clause 120, or the combined pharmaceutical dose form according to any one of clauses 121 to 123, the combination pack according to clause 124, the compound of formula (I) as defined in any one of clauses 1 to 119, the compound according to formula (Ia) as defined in any one of clauses 1 to 119 for the treatment and/or prevention of a disease or disorder, preferably selected from the diseases listed in clauses 126 and/or 127.
129. The pharmaceutical formulation according to any one of clauses 1 to 119 for producing a medicament for treatment and/or prevention of a disease or disorder, preferably selected from the diseases listed in clauses 126 and/or 127.
130. Method of treatment and/or prevention of a disorder and/or disease, preferably selected from the diseases listed in clauses 126 and/or 127, comprising administering the pharmaceutical formulation according to any one of clauses 1 to 119, the medicament according to clause 120, or the combined pharmaceutical dose form according to any one of clauses 121 to 123, the combination pack according to clause 124, the compound of formula (I) as defined in any one of clauses 1 to 119, the compound according to formula (Ia) as defined in any one of clauses 1 to 119.
131. A method for the preparation of the pharmaceutical formulation according to any one of clauses 1 to 119, comprising the following steps:
   step 1. Providing components a, b, c and d; and
   step 2. Mixing the components provided in step 1;
   whereby the following pharmaceutical formulation according to any one of clauses 1 to 119 is obtained.
132. The method according to clause 131, wherein the method further comprises step 3 and/or step 4: and/or step 5
   step 3. Adjusting the pH of the pharmaceutical formulation to a pH of 3 to 5; and/or
   step 4. Adjusting the osmolarity of the pharmaceutical formulation to an osmotic concentration between 150-450 mosmol/l;
   wherein step 3 can be carried before, during and/or after step 1, 2 and/or step 4; and/or wherein step 4 can be carried before, during and/or after step 1, 2 and/or step 3.
133. The method according to any one of clauses 131 to 132, wherein the method comprises the following steps
   providing an aqueous formulation of PEG-ADM, which comprises citric acid and optionally at least one pH regulator to adjust the pH to 3.5 and 4.5,
   followed by concentration of the aqueous formulation of PEG-ADM and
   subsequently reconstitution/dilution of the concentrated product by adding a solution of citric acid and/or sodium citrate, optionally at least one pH regulator and an osmolarity regulator and water, and
   wherein the pharmaceutical formulation has an osmotic concentration between 150-450 mosmol/L; and wherein the pH of the resulting aqueous formulation is between 3.5 and 4.5.
134. The method according to any one of clauses 131 to 133, wherein the method comprises the following steps
   providing an aqueous formulation of PEG-ADM, which comprises citric acid and optionally at least one pH regulator to adjust the pH to 3.5 and 4.5,
   providing citric acid and/or sodium citrate, optionally at least one pH regulator and an osmolarity regulator and
   mixing the solutions provided, and
   wherein the pharmaceutical formulation has an osmotic concentration of between 150-450 mosmol/l; and wherein the pH of the resulting aqueous formulation is between 3.5 and 4.5.
135. The method according to clause according to any one of clauses 131 to 134, wherein the method further comprises step 5
   Step 5 at least partially freezing the pharmaceutical formulation obtained after any one of steps 1, 2, 3 and/or 4; wherein step 4 can be carried before, during and/or after step 1, 2, 3 and/or step 4.
136. The formulation according any one of clauses 1 to 119 obtainable by the method according to any one of clauses 131 to 135.

### DESCRIPTION OF THE FIGURES

FIG. 1 shows a DSC of Example 1. The preparation of the Example is described in section B-1 below. The DSC method is described in section C-1 below. The results are described in section D-1 below.

FIG. 2 shows a DSC of Example 8. The preparation of the Example is described in section B-1 below. The DSC method is described in section C-1 below. The results are described in section D-1 below.

In FIG. 3, the aggregation and degradation over a time period of 24 months of Example 1-batch 1, Example 1-batch 2 and Example 8 are shown (method as described in section C-2 “SEC-HPLC for Purity, Monomer Portion”).

In FIG. 4, purity analysis using the “RP-HPLC for Assay of PEG-ADM” as described in section C-3 (for the quantitation and identification of PEG-ADM as well as the related substances and degradation products) is depicted.

### EXAMPLES

The following working examples illustrate the invention. The invention is not restricted to the examples. The percentages in the following tests and examples are, unless stated otherwise, percentages by weight; parts are parts by weight. Solvent ratios, dilution ratios and concentration data for the liquid/liquid solutions are each based on volume.

For all examples described below, a 40 kDa PEG-ADM was used (cf. compound according to formula (Ia)). Approx. 7.7 mg of this 40 kDa PEG-ADM equal to 1 mg ADM.

### A. Abbreviations

| | |
|---|---|
| ADM | adrenomedullin (human) |
| DSC | differential scanning calorimetry |
| FPF | Fine particle fraction |
| GSD | geometric standard deviation |
| PEG | polyethylene glycol |
| p.a. | pro analysis |
| q.s. | Quantum satis |
| VMD | volume median diameter |

Nomenclature of amino acids and peptide sequences is according to:

International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624

| Trivial Name | Symbol | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### B Preparation of Citric Acid- and Sodium Citrate-Supplemented PEG-ADM Solutions B-1 Preparation of a Pharmaceutical Formulation Comprising Components a to d

Different pharmaceutical formulations (Examples 1 to 13) comprising components a to d were prepared. For all examples described below, a the compound according to formula (Ia) was used (a 40 kDa PEG-ADM). Approx. 7.7 mg of this 40 kDa PEG-ADM equal to 1 mg ADM. The composition of the pharmaceutical formulations and the resulting concentrations of PEG-ADM [ADM] comprised in the final pharmaceutical formulations is listed in tables 1-1 and 1-2 below:

Table 1-1 and table 1-2 show the composition of Examples 1 to 8 and Examples 9 to 13, respectively. The concentration of ADM comprised in PEG-ADM is given in squared brackets. When referring to PEG-ADM the compound of formula (Ia) was used. In the PEG-ADM batch used, approx. 7.7 mg PEG-ADM comprised approx. 1 mg ADM (see e.g. Example 8 having a concentration of 7.7 mg/mL PEG-ADM, which equals to a concentration of 1 mg/mL ADM). “HCl” means hydrochloric acid 10% (m/V). Hydrochloric acid 10% was used to adjust the pH to approx. 4. “q.s.” means “quantum satin” for adjusting the pH to approx. 4.

TABLE 1-1
The PEG-ADM used was the compound according to formula (Ia). The concentration of ADM
comprised int the PEG-ADM is given in squared brackets ([ADM concentration]).

| | Examples | | | | | | | |
|---|---|---|---|---|---|---|---|---|
| | 1 | 2 | 3 | 4 | 5 | 6 | 7 | 8 |
| | [mg/mL] | [mg/mL] | [mg/mL] | [mg/mL] | [mg/mL] | [mg/mL] | [mg/mL] | [mg/mL] |
| PEG- | 3.696 | 0.385 | 0.77 | 1.54 | 3.08 | 4.62 | 6.16 | 7.7 |
| ADM | [0.48] | [0.05] | [0.1] | [0.2] | [0.4] | [0.6] | [0.8] | [1] |
| [ADM] | | | | | | | | |
| Citric | 5.380 | 5.380 | 5.380 | 5.380 | 5.380 | 5.380 | 5.380 | 5.380 |
| acid | | | | | | | | |
| anhydrous | | | | | | | | |
| Sodium | 2.242 | 2.415 | 2.406 | 2.388 | 2.351 | 2.315 | 2.278 | 2.242 |
| hydroxide | | | | | | | | |
| Sodium | 6.540 | 8.850 | 8.700 | 8.400 | 7.800 | 7.200 | 6.600 | 6.000 |
| chloride | | | | | | | | |
| HCl | q.s. | q.s. | q.s. | q.s. | q.s. | q.s. | q.s. | q.s. |

TABLE 1-2
The PEG-ADM used was the compound according to formula
(Ia). The concentration of ADM comprised int the PEG-
ADM is given in squared brackets ([ADM concentration]).

| | Examples | | | | |
|---|---|---|---|---|---|
| | 9 | 10 | 11 | 12 | 13 |
| | [mg/mL] | [mg/mL] | [mg/mL] | [mg/mL] | [mg/mL] |
| PEG-ADM | 0.2 | 0.385 | 0.77 | 10 | 23.1 |
| [ADM] | [0.026] | [0.05] | [0.10] | [1.30] | [3.00] |
| Citric acid | 5.380 | 5.380 | 5.380 | 5.380 | 5.380 |
| anhydrous | | | | | |
| Sodium | 2.242 | 2.242 | 2.242 | 2.242 | 2.242 |
| hydroxide | | | | | |
| Sodium | 6.540 | 6.540 | 6.540 | 6.540 | 6.540 |
| chloride | | | | | |
| HCl | q.s. | q.s. | q.s. | q.s. | q.s. |

For the preparation of Examples 1 to 13 a buffer solution and stock solution comprising PEG-ADM (Bayer AG, Germany) solution were utilized to prepare the pharmaceutical formulation according to Examples 1 to 13. The final pH of Examples 1 to 13 was approx. 4. The pH was determined via a titrator excellence T5, Mettler Toledo.

In the following, the preparation for Example 1 is described. For the preparation of Example 1, a stock buffer solution and stock solution comprising PEG-ADM (Bayer AG, Germany) solution were utilized. The final pH of Example 1 was approx. 4. The osmotic concentration was approx. 300 mosmol/l.

Stock Buffer solution: A stock buffer solution having a pH of 4.0 was prepared as follows: A vessel was filled with water and was mixed with 5.8877 g of citric acid monohydrate and 2.3203 g of sodium hydroxide. The pH was adjusted with 6.3 mL of hydrochloric acid to 4.0. The solution was filled in a volumetric flask and water was added to 1000 mL. 500 mL of the buffer were mixed with 4.50 g of sodium chloride. To adjust the pH to 4.0 1.3 mL of sodium hydroxide 1N was added.

Stock solution comprising PEG-ADM: PEG-ADM stock solution had a concentration of 7.7 mg/mL of PEG-ADM (comprising 1 mg/mL ADM).

Example 1: For Example 1, 9.6 mL of the PEG-ADM stock solution was mixed with 10.4 mL of stock buffer solution obtaining a solution of 0.48 mg/mL ADM.

Example 2: For Example 2, 1 mL of the PEG-ADM stock solution was mixed with 19 mL of stock buffer solution obtaining a solution of 0.05 mg/mL ADM.

Examples 3 to 11 were prepared accordingly.

The stock solution used for Examples 12 and 13 had a concentration of 31.26 mg/mL of PEG-ADM (comprising 4.06 mg/mL ADM).

B-2 Viscosity

The viscosity of Examples 1 to 13 was determined as follows: The viscosity is determined by an automatic rolling ball viscometer method according to Ph.Eur. 2.2.49 (2018), using an Anton Paar AMVn Automated Microviscometer.

The results of the measurement are listed in tables 2-1 and 2-2 below:

TABLE 2-1

| | Viscosity of Examples 1 to 7 and buffer | | | | | | | |
|---|---|---|---|---|---|---|---|---|
| Example | buffer | 1 | 2 | 3 | 4 | 5 | 6 | 7 |
| viscosity | approx. | approx. | 1.0722 | 1.1029 | 1.1666 | 1.2927 | 1.4325 | 1.5440 |
| [mPa*s] | 1.063 | 1.2413 | | | | | | |

TABLE 2-2

| | Viscosity of Examples 8 to 13 | | | | | |
|---|---|---|---|---|---|---|
| Example | 8 | 9 | 10 | 11 | 12 | 13 |
| viscosity | 1.9969 | 1.0107 | 1.0228 | 1.0550 | 1.9176 | 3.7541 |
| [mPa*s] | | | | | | |

### B-3 Preparation the PEG-ADM Solution for DSC Analysis

The PEG-ADM solution for DSC analysis was prepared by thawing Example 1 and 8 (cf. section B-1 above) containing approx. 3.696 and 7.7 mg/mL PEG-ADM respectively (equivalent to 0.48 mg/mL and 1 mg/mL ADM comprised in the PEG-ADM) in a citrate buffer having a pH of 4, and mixing the thawed solution with a formulation buffer containing sodium chloride in a citrate buffer pH 4. The resulting solution was stirred, filtered through a pre-filter and sterilizing filter, and filled aseptically into vials which were subsequently closed with an injection stopper and sealed with a pharmaceutical cap. The vials with formulated PEG-ADM solution were frozen to <−15° C.

The samples for DSC analysis of Examples 9-13 were prepared in the same way as the solutions described in section B.1.

### C Analysis: Methods

This section C describes the general methods used for the analysis of the pharmaceutical formulation. The results of this analysis are described and discussed in section D below.

The DSC method as described in section C-1 was used to for thermionic characterization of the pharmaceutical formulation.

The stability of the pharmaceutical formulation was also analyzed. The stability analysis included the investigation of potential aggregation and degradation of the pharmaceutical formulation, in particular for PEG-ADM. In particular, the “SEC-HPLC for Purity, Monomer Portion” as described in section C-2 was used to determine the amount of PEG-ADM (monomer portion) and/or HMV aggregates (high molecular weight aggregates) were built during a certain time period. The monomer portion is the intended form of PEG-ADM, formation of HMW indicates aggregation of PEG-ADM molecules with formation of dimers or higher aggregates and, thus, is an indicator that the pharmaceutical formulation is unstable. The stability over 24 months at a storage temperature of −20° C. was investigated. The “RP-HPLC for Assay of PEG-ADM” as described in section C-3 was used for the quantitation and identification of PEG-ADM as well as the related substances and degradation products.

A freeze thaw cycling study as described in section C-4 was performed to investigate the stability of the pharmaceutical formulation after repeated freeze thaw cycles.

The nebulization properties were analyzed using the method described in section C-5.

### C-1 DSC Method

Example 1 and 8 (cf. section B-1 above) were analyzed. Differential Scanning calorimetry measurements were performed using a TA Instruments Q2000 DSC with Universal Analysis software. Two different temperature profiles were applied in order to differentiate between slow freezing which results in partial or complete crystallization of sodium chloride, and fast freezing which prohibited crystallization.

The slow freezing method comprised loading the TZero pan with the sample and an empty reference pan into the measuring cell at room temperature, followed by ramping the temperature to −30° C. at 5.0° C./min. The temperature was maintained at −30° C. for 15 minutes, followed by a further ramp to −80° C. at 10° C./min and holding the sample at −80° C. for 10 minutes. After the isothermal hold the temperature was increased at 10° C./min to +10° C.

The fast freezing method comprised loading the TZero pan with the sample and an empty reference pan into the measuring cell at room temperature, followed by ramping the temperature to −80° C. at 10° C./min and holding the sample at −80° C. for 10 minutes. After the isothermal hold the temperature was increased at 10° C./min to +10° C.

The samples according to Example 9-13 were prepared and analyzed via DSC using two methods identically to the procedure described above.

### C-2 Method: SEC-HPLC for Purity of PEG-ADM

Examples 1 and 8 (cf. section B-1 above) as well as Examples 9-13 were analyzed. HPLC, size exclusion chromatography (SEC-HPLC) with UV detection at 280 nm analysis via 100% method comparing peak areas. The separation and quantitation of PEG-ADM (the monomer portion) as well as the dimer and the HMW aggregates (high molecular weight aggregates) are conducted by SEC-HPLC on an SEC-column using the 100% area method. (Ph. Eur., 2.2.29 (2015), USP<621> (2011)).

Mobile phase is prepared from NaH₂PO₄ monohydrate, NaCl p.a., Water for chromatography, Ethanol HPLC grade, and 25 mM Citrate buffer pH 4.0.

As stationary phase, e.g. Wyatt SEC Protein Column WTC-03055 with 300 mm length and 7.8 mm inner diameter can be used. An isocratic elution with a flow of 0.5 mL/min is applied at a temperature of 22° C. and a run time of 30 min, the injection volume is 50 μL.

### C-3 Method: RP-HPLC for Assay of PEG-ADM

Example 1 and 8 (cf. section B-1 above) were analyzed. The separation, quantitation and identification of PEG-ADM as well as the related substances and degradation products are conducted by RP-HPLC on a reversed phase column using an external standard method or by 100% area method with UV detection at 280 nm or 210 nm, resp. (Ph. Eur., 2.2.29 (2015), USP<621> (2011)).

Mobile phase is prepared from trifluoracetic acid >99.0%, acetonitrile for chromatography, water for chromatography, and 25 mM citrate buffer. A gradient between 0.1% TFA in water for chromatography and 0.1% TFA in acetonitrile for chromatography is applied. As stationary phase, e.g. YMC-Triart Bio C4 with 150 mm length and 3.0 mm inner diameter can be used. The column temperature was 40° C. and the run time was 30 minutes, the injection volume was 50 μL.

### C-4 Method: Thaw Cycling Study (Freeze Thaw Protocol)

A sample of Example 8 was frozen at −70° C. and thawed at room temperature. This cycle was repeated five times. The time schedule is depicted in the following table 3-1.

TABLE 3-1
Time schedule

| | Time in freezer at −70° C. | Time at room temperature |
|---|---|---|
| Cycle | [minutes] | [minutes] |
| 1 | 33 | 56 |
| 2 | 24 | 68 |
| 3 | 38 | 77 |
| 4 | 37 | 73 |
| 5 | 30 | 65 |

Before each freezing step a sample of 1.5 mL was taken and stored in cryo vial at 2-8° C. until analysis.

Samples according to Example 9 to 13 with a fill volume of 2.28 mL were frozen to −70° C. and thawed at room temperature. The cycle was repeated five times. The time schedule is depicted in the following table 3-2.

TABLE 3-2
Time schedule

| | Thawing Cycle [time in min] | | | | |
|---|---|---|---|---|---|
| Example | 1 | 2 | 3 | 4 | 5 |
| 9 | 32 | 29 | 26 | 26 | 24 |
| 10 | 32 | 29 | 26 | 27 | 23 |
| 11 | 33 | 29 | 25 | 28 | 23 |
| 12 | 31 | 29 | 25 | 28 | 23 |
| 13 | 28 | 29 | 25 | 25 | 23 |

### C-5 Method: Determination of Nebulization Properties

Example 1 was frozen and re-thawed. This solution was nebulized and measured using three different Aerogen® Solo nebulizer heads with Pro-X Controller. The droplet size measurements were performed with Sympatec HELOS laser diffraction.

Example 9-13 were frozen and re-thawed. The solutions were nebulized and measured using three different Aerogen® Solo nebulizer heads with Pro-X Controller. The droplet size measurements were performed with a Sympatec HELOS laser diffraction.

### D Results

### D-1 Results: DSC

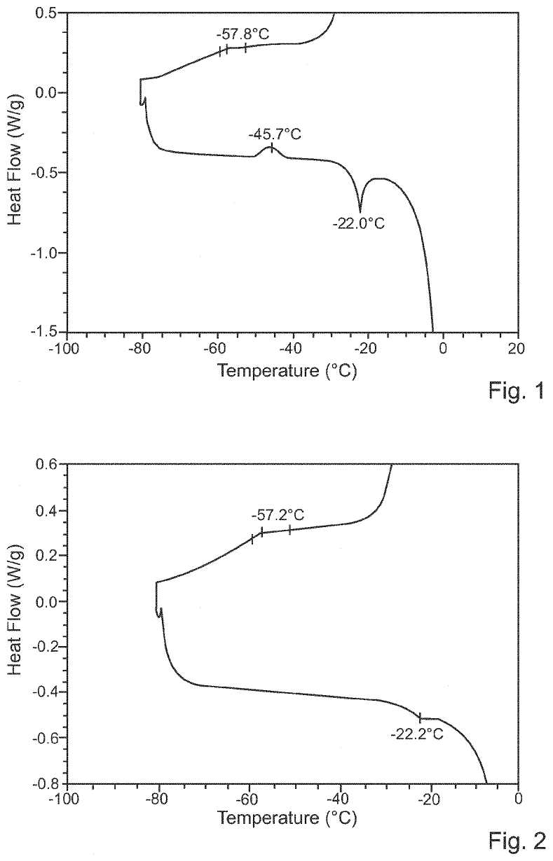

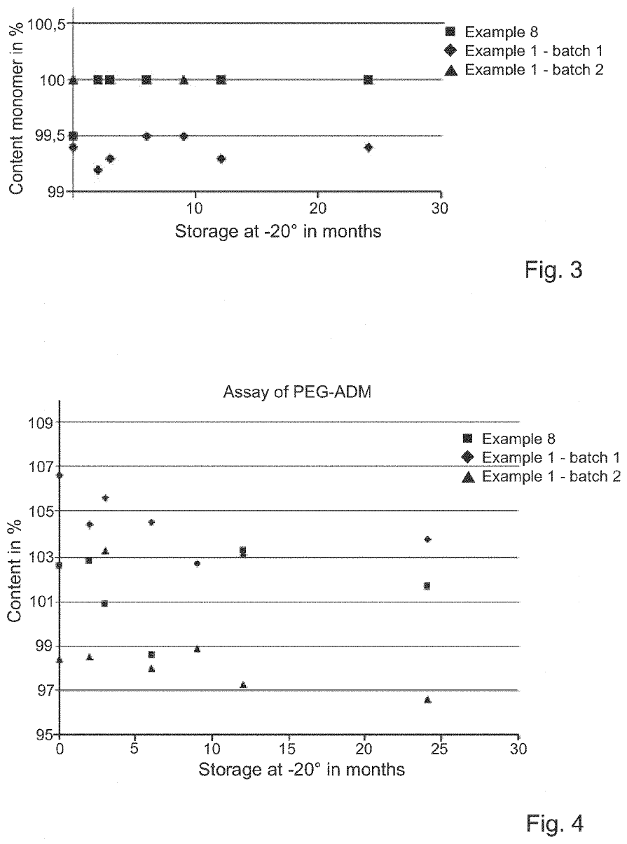

Examples 1 and 8 were analyzed. The DSC's are depicted in FIG. 1 (Example 1) and FIG. 2 (Example 8). The thermal characterization of the solution via Differential Scanning Calorimetry indicated a relatively low glass transition temperature of −58° C. for the solutes in amorphous state, and a eutectic temperature of −22° C. for the solution with completely crystallized sodium chloride. These thermal properties clearly suggest that the solution is not stable unless it is present in fully frozen state at a storage temperature of e.g. −58° C. or lower.

Examples 9-13 were analyzed. The results are overall well comparable with the results for Examples 1 and 8. The Examples 9, 10 and 11 also show a glass transition temperature during freezing at −58° C., a small crystallization peak around −52° C. and an endothermal peak around −22° C. which suggests a eutectic. The peak intensity differs for the thermal treatment methods applied.

The samples 12 and 13, which contain higher concentrations of PEG-ADM, show less pronounced crystallization and eutectic peaks during heating, which could suggest that the PEG-ADM content partially inhibits the crystallization of NaCl in the formulation.

Same as for Examples 1 and 8, these thermal properties suggest that the solutions of Examples 9 to 13 are not stable unless present in fully frozen state at a storage temperature of e.g. −58° C. or lower.

### D-2 Results: Stability Analysis

The stability of Example 1 and Example 8 (cf. section B-1 above) was observed over a time period of 24 months at a storage temperature of −20° C. Two batches of Example 1 (Example 1-batch 1; Example 1-batch 2) and one batch of Example 8 were analyzed. After 0, 2, 3, 6, 9 12 and 24 months, respectively, a sample of the respective batch was taken and analyzed with the methods described in section C-2 and C-3 above.

The stability results for Example 1-batch 1, Example 1-batch 2 and Example 8 are shown in FIGS. 3 and 4 below.

In FIG. 3, the aggregation of Example 1-batch 1, Example 1-batch 2 and Example 8 are shown (method as described in section C-2 “SEC-HPLC for Purity, Monomer Portion”). From the FIG. 3 it can be seen that over a time period of 24 months the amount of the monomer portion of PEG-ADM remains over 99%. Thus, only a small amount of HMW aggregates (high molecular weight aggregates) or dimers were built over the 24-months-period. This indicates that the pharmaceutical formulations show an excellent stability.

In FIG. 4, the “RP-HPLC for Assay of PEG-ADM” as described in section C-3 was used for the quantitation and identification of PEG-ADM as well as the related substances and degradation products. From the FIG. 4 it can be seen that over a time period of 24 months the respective samples show a PEG-ADM content over approx. 96% or in other words the respective samples show a loss of content of PEG-ADM of only 1 to 3%. Thus, only a small amount of related substances and degradation products were built over the 24-months-period. This indicates that the pharmaceutical formulations show an excellent stability.

In summary, the pharmaceutical formulations (examples 1 and 8) show a very good stability over the 24-months-period.

For Examples 9 to 13, the stability was observed over a 1-month-period. The storage over the 1-month-period and the analysis of the samples was performed as described for Example 1 and 8 above. The results are shown in table 3-3 below:

TABLE 3-3
Stability of Examples 9 to 13 over 1-month-period

| | | | | | 40 kDa | Sum of |
|---|---|---|---|---|---|---|
| Ex- | Time | Any | | | PEG- | dimer and |
| ample | point | unspec. | Sum | Adrenomedullin | ADM | HMW |
| 9 | Start | <0.05% | <0.05% | <0.05% | <0.05% | 100 |
| 9 | After | <0.05% | <0.05% | <0.05% | <0.05% | 100 |
| | 1 month | | | | | |
| 10 | Start | <0.05% | <0.05% | <0.05% | <0.05% | 100 |
| 10 | After | <0.05% | <0.05% | <0.05% | <0.05% | 100 |
| | 1 month | | | | | |
| 11 | Start | <0.05% | <0.05% | <0.05% | <0.05% | 100 |
| 11 | After | <0.05% | <0.05% | <0.05% | <0.05% | 100 |
| | 1 month | | | | | |
| 12 | Start | <0.05% | <0.05% | <0.05% | <0.05% | 100 |
| 12 | After | <0.05% | <0.05% | <0.05% | <0.05% | 100 |
| | 1 month | | | | | |
| 13 | Start | <0.05% | <0.05% | <0.05% | <0.05% | 100 |
| 13 | After | <0.05% | <0.05% | <0.05% | <0.05% | 100 |
| | 1 month | | | | | |

It can be seen from table 3-3 that Examples 9 to 13 show a very good stability over a 1-month-period.

### D-3 Results: Thaw Cycling Study

The thaw cycling stability of Example 8 and Examples 9 to 13 were investigated. To assess potential aggregation and degradation SEC- and RP-HPLC (methods described in sections C-2 and C-3) was performed. The results before and after the five freeze thaw cycles are shown in the following tables 4-1 (Example 8) and table 4-2 (Examples 9 to 13).

TABLE 4-1
Test methods and results before and after
5 five freeze-thaw-cycles of Example 8

| | Before 5 Freeze | After 5 freeze/ |
|---|---|---|
| | Thaw Cycles | thaw cycles |
| Purity (RP-HPLC; section C-3) | 99.72% | 99.65% |
| Adrenomedullin | 0.18% | 0.23% |
| 40 kDa PEG-ADM | <0.05% | <0.05% |
| Any unspecified impurity | 0.09% | 0.08% |
| Sum of all organic impurities | 0.27% | 0.31% |
| Purity, monomer portion | 99.34% | 99.30% |
| (SEC-HPLC; section C-2) | | |
| Sum of dimer and HMW | 0.66% | 0.70% |

The purity of Example 8 before the five thaw cycles was 99.72% (RP-HPLC; section C-3) and 99.34% (purity, monomer portion; SEC-HPLC; section C-2). The purity of Example 8 before the five thaw cycles was 99.65% (RP-HPLC; section C-3; difference to “before” 0.07%) and 99.30% (purity, monomer portion; SEC-HPLC; section C-2; difference to “before” 0.04%).

Therefore, after five cycles of freezing and thawing nearly no degradation or aggregation was observed. These results demonstrate that Example 8 shows an excellent stability even if frozen and re-thawed.

TABLE 4-2
Test methods and results before, during and after
5 five freeze-thaw-cycles of Examples 9 to 13.

| | | | | Purity, |
|---|---|---|---|---|
| | | | | monomer |
| | | Any | Sum of all | portion |
| | | unspecified | organic | (SEC-HPLC; |
| | Cycle | impurity. | impurities | section C-2) |
| Example 9 | Before 5 Freeze | <0.05% | <0.05% | 100% |
| | Thaw Cycles | | | |
| Example 9 | After 5 freeze/ | <0.05% | <0.05% | 100% |
| | thaw cycles | | | |
| Example 10 | Before 5 Freeze | <0.05% | <0.05% | 100% |
| | Thaw Cycles | | | |
| Example 10 | After 5 freeze/ | <0.05% | <0.05% | 100% |
| | thaw cycles | | | |
| Example 11 | Before 5 Freeze | <0.05% | <0.05% | 100% |
| | Thaw Cycles | | | |
| Example 11 | After 5 freeze/ | <0.05% | <0.05% | 100% |
| | thaw cycles | | | |
| Example 12 | Before 5 Freeze | <0.05% | <0.05% | 100% |
| | Thaw Cycles | | | |
| Example 12 | After 5 freeze/ | <0.05% | <0.05% | 100% |
| | thaw cycles | | | |
| Example 13 | Before 5 Freeze | <0.05% | <0.05% | 100% |
| | Thaw Cycles | | | |
| Example 13 | After 5 freeze/ | <0.05% | <0.05% | 100% |
| | thaw cycles | | | |

The results confirm that Examples 9 to 13 have an excellent stability even if frozen and re-thawed several times.

### D-4 Nebulization Experiments

The results of the nebulization properties of Example 1 are shown in table 5 below. A sample of Example 1 was nebulized with three different Aerogen® Solo nebulizer heads with Pro-X Controller (cf. nebulizer batch no. 1, 2 and 3 in table 5).

TABLE 5
Nebulization properties, expressed as volume median diameter
(VMD in micrometer (μm)), of Example 1 (frozen and re-
thawed formulation). GSD means geometric standard deviation.
“output rate” in gram per minute (g/min) indicates
how many grams of solution are nebulized per minute (throughput).
“FPF” in percent (%) means fine particle fraction,
which indicates the percentage of particles/droplets below 5 μm.

| Nebulizer | VMD | | Output rate | FPF |
|---|---|---|---|---|
| batch no. | [μm] | GSD | [g/min] | [%] |
| 1 | 5.1 | 1.8 | 0.25 | 58.5 |
| 2 | 5.0 | 1.7 | 0.19 | 58.8 |
| 3 | 5.3 | 1.7 | 0.20 | 55.4 |

For each of the Aerogen Solo nebulizers it was possible to perform the nebulization of the PEG-ADM solution to droplets with the intended droplet size, a narrow geometric standard deviation, and with an output rate of solution of that enables nebulization of 1 mL solution within 4 to 5 minutes. The fine particle fraction was between 55% and 59% which is in agreement with the expected output of the device. The results confirm that the frozen and thawed PEG-ADM formulation can be nebulized using the Aerogen Solo device in a suitable manner for delivering PEG-ADM via inhalation.

The results of the nebulization properties of Example 9-13 are shown in table 6 below. A sample of Example 9 to 13 was nebulized with three different Aerogen® Solo nebulizer heads per formulation with Pro-X Controller.

TABLE 6
Nebulization properties, expressed as volume median diameter
(VMD in micrometer (μm)), of Examples 9 to 13 (frozen and
re-thawed formulation). GSD means geometric standard deviation.
“output rate” in gram per minute (g/min) indicates
how many grams of solution are nebulized per minute (throughput).
“FPF” in percent (%) means fine particle fraction,
which indicates the percentage of particles/droplets below 5 μm.

| | | VMD | | Output rate | FPF |
|---|---|---|---|---|---|
| Example | Nebulizer no. | [μm] | GSD | [g/min] | [%] |
| 9 | 1 | 4.95 | 1.8 | 0.48 | 57.5 |
| 9 | 2 | 5.52 | 1.9 | 0.48 | 51.5 |
| 9 | 3 | 4.96 | 1.8 | 0.38 | 57.5 |
| 10 | 1 | 4.63 | 1.7 | 0.33 | 61.9 |
| 10 | 2 | 5.67 | 1.9 | 0.55 | 49.8 |
| 10 | 3 | 5.85 | 1.9 | 0.52 | 48.0 |
| 11 | 1 | 5.30 | 1.9 | 0.54 | 55.1 |
| 11 | 2 | 5.38 | 1.8 | 0.54 | 52.6 |
| 11 | 3 | 6.01 | 1.9 | 0.40 | 46.6 |
| 12 | 1 | 9.04 | 2.2 | 0.12 | 37.4 |
| 12 | 2 | 6.70 | 1.9 | 0.12 | 52.7 |
| 12 | 3 | 12.00 | 2.6 | 0.14 | 32.1 |

| | |
|---|---|
| 13 | Nebulization not possible for Example 13 |

For Example 9-11, it was possible to perform multiple nebulization of the PEG-ADM solution to droplets with the intended droplet size, a narrow geometric standard deviation, and with an output rate of solution that enables nebulization of 1 mL solution within 2-3 minutes. The fine particle fraction was between 47% and 62% which is in agreement with the expected output of the device. The results confirm that the frozen and thawed PEG-ADM formulation described in Example 9-11 can be nebulized using the Aerogen® Solo device in a suitable manner for delivering PEG-ADM via inhalation.

For Example 12, it was possible to perform the nebulization of the PEG-ADM solution to droplets, but the droplet size increased considerably over the nebulization process, and the geometric standard deviation was higher than for the less concentrated solutions according to Examples 9-11. This also impacted the fine particle fraction which was reduced, as well as the throughput.

For Example 13, it was not possible to generate droplets via the Aerogen® Solo nebulizer, as the solution was too viscous to pass through the nebulizer membrane. Therefore, the nebulization properties could not be determined.

1. A liquid pharmaceutical formulation comprising:
0.04 mg/mL to 145 mg/mL of PEG-ADM, wherein the PEG-ADM is a compound according to the general formula (I), 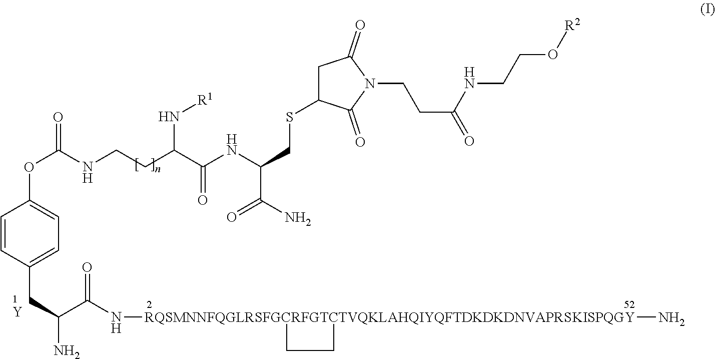
in which:
n represents the number 0, 1, 2, or 3,
R¹ represents hydrogen, methyl, ethyl, n-propyl, or isopropyl, and
R² represents linear or branched PEG 20 kDa to 80 kDa endcapped with a methoxy-group,
or a hydrate thereof, solvate thereof, salt thereof, pharmaceutically acceptable salt thereof, or the solvates of salts thereof;
a solvent;
a pH regulator; and
an osmolarity regulator;
wherein the pharmaceutical formulation has a pH of 3 to 5; and wherein the osmolar concentration is between 150 to 450 mosmol/L, and
wherein the concentrations of components are based on the total volume of the liquid pharmaceutical formulation.

2. The pharmaceutical formulation of claim 1, wherein the pharmaceutical formulation is a solution, a dispersion, a frozen dispersion, a frozen solution and/or an aqueous solution.

3. The pharmaceutical formulation of claim 1, wherein the PEG-ADM is selected from compounds of the formula (I) in which
n represents the number 1 or 2,
R¹ represents hydrogen or methyl,
R² represents linear PEG 40 kDa endcapped with a methoxy-group.

4. The pharmaceutical formulation of claim 1, wherein the solvent is selected from the group of water, a buffer, sodium chloride solution, solution of citric acid, solution of citric acid anhydrous, solution of citric acid monohydrate, hydrochloric acid, sodium hydroxide solution, sodium citrate solution, and/or mixtures.

5. The pharmaceutical formulation of claim 1, wherein the pharmaceutical formulation comprises 0.1 mg/mL to 250 mg/mL of the pH regulator.

6. The pharmaceutical formulation of claim 1, wherein the pH regulator comprises or is citric acid, a salt of citric acid, a pharmaceutical acceptable salt of citric acid, a derivative of citric acid, and/or mixtures thereof.

7. The pharmaceutical formulation of claim 1, wherein the osmolarity regulator is selected from the group consisting of sodium chloride, citric acid, a salt, pharmaceutical acceptable salt, derivative of citric acid and/or mixtures thereof.

8. The pharmaceutical formulation of claim 1, wherein the pharmaceutical formulation comprises as pH regulator:
0.1 mg/mL to 100 mg/mL citric acid;
0.01 mg/mL to 50 mg/mL sodium hydroxide;
0.1 mg/mL to 100 mg/mL hydrochloric acid.

9. The pharmaceutical formulation of claim 1, wherein the pharmaceutical formulation comprises 0.01 mg/mL to 100 mg/mL of the osmolarity regulator.

10. The pharmaceutical formulation of claim 1, wherein the pharmaceutical formulation comprises:
0.5 mg/mL to 25 mg/mL of the pH regulator, and
0.1 mg/mL to 30 mg/mL of the osmolarity regulator.

11. The pharmaceutical formulation of claim 1, wherein the pharmaceutical formulation is for inhalation.

12. Medicament comprising the pharmaceutical formulation of claim 1 or a medicament comprising the pharmaceutical formulation of claim 1 in combination with an inert nontoxic pharmaceutically suitable excipient, optionally in combination with a further active ingredient.

13. The pharmaceutical formulation of claim 1, wherein the pharmaceutical formulation is for use in the treatment and/or prevention of diseases and/or disorders.

14. The pharmaceutical formulation of claim 13, wherein the disease and/or disorder is selected from
pulmonary disorders, such as pulmonary hypertension; secondary pulmonary hypertension; pulmonary hypertension following pulmonary embolism with and without acute cor pulmonale; primary pulmonary hypertension; chronic obstructive pulmonary disease; asthma; acute pulmonary edema; chronic pulmonary edema; allergic alveolitis; pneumonitis due to inhaled organic dust; pneumonitis due to inhaled particles of fungal, actinomycetic or other origin; acute chemical bronchitis; acute chemical pulmonary edema and/or chronic chemical pulmonary edema (e.g. after inhalation of phosgene, nitrogen oxide); neurogenic pulmonary edema; acute pulmonary manifestations due to radiation; chronic pulmonary manifestations due to radiation; acute and/or chronic interstitial lung disorders (such as but not restricted to drug-induced interstitial lung disorders, e.g. secondary to Bleomycin treatment); acute lung injury (ALI); acute lung injury (ALI) in adult or child including newborn; acute respiratory distress syndrome (ARDS); acute respiratory distress syndrome (ARDS) in adult or child including newborn; ALI/ARDS secondary to pneumonia and sepsis, aspiration pneumonia and ALI/ARDS secondary to aspiration (such as but not restricted to aspiration pneumonia due to regurgitated gastric content); ALI/ARDS secondary to smoke gas inhalation; transfusion-related acute lung injury (TRALI), ALI/ARDS or acute pulmonary insufficiency following surgery; trauma or burns, ventilator induced lung injury (VILI); lung injury following meconium aspiration; pulmonary fibrosis; and mountain sickness;
ALI/ARDS secondary to pneumonia caused by bacterial infection of the lungs, such as, but not restricted to, bacterial pneumonia caused by Pneumococci, Haemophilus Influenzae, Mycoplasma pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria;
ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV);
ALI/ARDS secondary to pneumonia caused by fungal infections such as, but not restricted to, fungal pneumonia caused by Pneumocystis Jirovecii;
ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP);
ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue);
ALI/ARDS secondary to pneumonia occurring in consequence of bacterial and/or virus infection;
ALI/ARDS secondary to pneumonia occurring in consequence of a bacterial superinfection of a primary lung affection by viruses and
prevention and/or treatment of lung dysfunction after lung transplantations.

15. A method for the preparation of the pharmaceutical formulation of claim 1, comprising:
providing 0.04 mg/mL to 145 mg/mL of the PEG-ADM, the solvent, the pH regulator, and the osmolarity regulator; and
mixing the 0.04 mg/mL to 145 mg/mL of the PEG-ADM, the solvent, the pH regulator, and the osmolarity regulator;
whereby the pharmaceutical formulation of claim 1 is obtained.


SEQUENCE LISTING (2 sequences)

SEQ ID NO: 1 - protein, 51 aa, Artificial Sequence
  Adrenomedullin 2 - 52
  DISULFID (15)..(20)
  MOD_RES (51)..(51): AMIDATION
RQSMNNFQGLRSFGCRFGTCTVQKLAHQIYQFTDKDKDNVAPRSKISPQGY

SEQ ID NO: 2 - protein, 52 aa, Artificial Sequence
  modified Adrenomeddulin 1 - 52
  MOD_RES (1)..(1): modified Tyrosine residue
  DISULFID (16)..(21)
  MOD_RES (52)..(52): AMIDATION
YRQSMNNFQGLRSFGCRFGTCTVQKLAHQIYQFTDKDKDNVAPRSKISPQGY